(19) United States
(12) Patent Application Publication
(10) Pub. No.: US 2024/0115178 A1
(43) Pub. Date: Apr. 11, 2024

(54) SYSTEMS AND METHODS FOR NEURAL INTERFACES

(71) Applicant: Precision Neuroscience Corporation, New York, NY (US)

(72) Inventors: Benjamin I. RAPOPORT, New York, NY (US); Demetrios Philip Papageorgiou, Weston, MA (US); Mark Hettick, Fremont, CA (US); Adam Poole, Brooklyn, NY (US); Elton Ho, San Mateo, CA (US)

(73) Assignee: PRECISION NEUROSCIENCE CORPORATION, New York, NY (US)

(21) Appl. No.: 18/380,918

(22) Filed: Oct. 17, 2023

Related U.S. Application Data
(63) Continuation of application No. 18/148,656, filed on Dec. 30, 2022.
(60) Provisional application No. 63/295,795, filed on Dec. 31, 2021.

(51) Int. Cl. A61B 5/25

(52) U.S. Cl. CPC A61B 5/25; A61B 5/163

(57) ABSTRACT

Disclosed herein are systems and methods for neural interfaces. Neural interfaces may form minimally invasive and high-scalable bidirectional brain-computer interfaces, which may be used in the treatment of a variety of disorders of the brain and nervous system. Disclosed are methods for a minimally invasive technique for implanting neural interfaces, a neural interface configured to be placed between the brain and the dura and configured to record from and/or stimulate the cortical surface. Also disclosed are methods for attaching a plurality of microelectrode arrays to form a neural interface device, and fabricating neural interfaces including microelectrode arrays and pockets to facilitate their insertion. The disclosed systems and methods also include neural decoding techniques.

### PRIORITY

The present application is a continuation of U.S. application Ser. No. 18/148,656, filed Dec. 30, 2022, which claims priority to U.S. Provisional Patent Application No. 63/295,795, titled SYSTEMS AND METHODS FOR NEURAL INTERFACES, filed Dec. 31, 2021, which are hereby incorporated by reference herein in their entireties.

### TECHNICAL FIELD

The present disclosure is directed towards neural interfaces, including brain-computer interfaces.

### BACKGROUND

Neural recording and stimulation techniques, such as those related to brain-computer interfaces, involve design trade-offs among (1) spatial resolution, (2) temporal resolution, (3) degree of invasiveness and collateral damage to normal brain tissue, and (4) optimization for electrical recording and/or electrical stimulation. Accordingly, there remains a need for high-bandwidth neural interfaces for the brain that allow for improved spatial resolution, temporal resolution, reduced collateral damage to the normal brain tissue and optimization for electrical recording and/or electrical stimulation.

Conventional techniques for recording and/or stimulating the nervous system face many challenges. For example, imaging techniques such as magnetic resonance imaging (MRI) and computed tomography (CT) provide non-invasive methods for examining brain tissue. However, these non-invasive imaging techniques are unable to detect all functional lesions, do not provide a method for imaging electrical activity in the nervous system, lack temporal resolution, and are unable to provide a mechanism for therapeutic electrophysiologic intervention.

Electromagnetic recording techniques such as electroencephalography (EEG) and magnetoencephalography (MEG) are non-invasive and provide temporal resolution of electrical activity in the brain. However, the resolution of such techniques is limited, due to both the physical distance separating the electrodes from the brain, and by the dielectric properties of the scalp and skull. Accordingly, there is a need for improved recording of neural activity with improved spatial resolution.

Additional techniques such as electrocorticography (ECoG) or intracranial EEG include forms of electroencephalography that provide improved spatial resolution by placing electrodes directly onto the cortical surface of the brain. However, the improved spatial resolution is conventionally achieved only by way of a highly invasive surgical procedure, a craniotomy, which requires the temporary surgical removal of a significant portion of the skull.

Another technique for recording and/or stimulating involves the use of depth electrodes which are capable of recording electrical activity in the nervous system with high spatial and temporal resolution. However, conventional depth electrodes are limited in that they are only able to record from a small volume or tissue, or a small population of neurons. Additionally, the placement of depth electrodes is highly invasive, and results in damage or destruction of normal brain tissue including neurons. Accordingly, the number of depth electrodes that can be safely placed is limited, as is the ability to adjust the spatial placement of the electrodes once they are placed, but for the minor adjustments to the depth of the electrode at the time of placement.

Another technique for recording and/or stimulating the nervous system includes the use of deep brain stimulation (DBS) electrodes, which may be configured to stimulate brain regions with millimetric and sub-millimetric precision. Although the DBS electrodes may be implanted leveraging minimally invasive surgical techniques, the electrode penetrates the brain which results in damage to the brain and carries risks including hemorrhage, stroke, and seizures. DBS electrodes can be used for stimulation as a way of treating conditions such as Parkinson's disease and essential tremor, and potentially some forms of epilepsy. However, the number of DB S electrodes that can safely be placed is limited, as is the ability to adjust the spatial placement of the electrodes, once they are placed. In practice, DBS techniques have an excellent safety profile demonstrated over two decades of standard clinical use, but as these electrodes are macroscopic, only a small number (typically one or two) are placed in any single patient.

Brain-computer interfaces have shown promise as systems for restoring, replacing, and augmenting lost or impaired neurological function in a variety of contexts, including paralysis from stroke and spinal cord injury, blindness, and some forms of cognitive impairment. Progress in the development of neural interfaces has been made over the past several decades, including in the areas of applied neuroscience and multichannel electrophysiology, mathematical and computation approaches to neural decoding, power-efficient custom electronics and the development of application-specific integrated circuits, as well as materials science and device packaging. Nevertheless, the practical impact of such systems remains limited, even for the small number of patients worldwide who have received highly customized implants and interfaces through involvement in carefully monitored clinical trials. Conventional systems for brain-computer interfaces are limited in that the surgical procedures involved in implanting the neural interfaces are extensive and may cause irreversible damage to the brain tissue, similar to what is discussed above. Further, conventional systems for brain-computer interfaces may not be capable of integrating large amounts of neural data, spanning across multiple brain regions.

### SUMMARY

Disclosed herein are neural interfaces for the brain, including brain-computer interfaces.

Progress toward the development of brain-computer interfaces has signaled the potential to restore, replace, or augment lost or impaired neurological function in a variety of disease states. As discussed above, existing brain-computer interfaces rely on invasive surgical procedures or brain-penetrating electrodes, which limit addressable applications of the technology and the number of eligible patients.

By contrast, the described systems, methods and related apparatus are configured to provide enhanced safety, lesser tissue damage, and less total procedural time, while providing higher and improved spatial resolution and channel counts. The disclosed systems, methods and related apparatus may involve the use of higher-spatial resolution micro-electrocorticography (μECoG) which provides improved signal quality while at the same time requiring minimal invasiveness.

Described are a modular and highly scalable system of conformable thin-film microelectrodes designed for minimally invasive deployment on the cortical surface. Disclosed are techniques for μECoG array fabrication and minimally invasive insertion.

Described herein are systems, methods and related apparatus for constructing a neural interface, including conformable thin-film electrode arrays and a minimally invasive surgical delivery system that together facilitate communication with large portions of the cortical surface in bidirectional fashion (enabling both recording and stimulation).

Additionally, described herein are in vivo experimental data and working examples of embodiments built in accordance with the present disclosure that demonstrate performance involving rapid implantation of thousands of electrodes simultaneously in multiple regions of the neocortex in both hemispheres, including areas related to vision and sensorimotor function. Also demonstrated are the use of the disclosed arrays for electrophysiologic functions required of contemporary brain-computer interfaces, including neural recording, cortical stimulation, and decoding.

The in vivo experimental data demonstrates the safety and feasibility of delivering reversible implants to multiple functional regions in both hemispheres of the Göttingen minipig brain simultaneously, without requiring a craniotomy, at an effective insertion rate faster than 40 ms per channel, without damaging the cortical surface even following implantation of more than 2,000 microelectrodes.

The disclosed systems are configured for high-density and high-channel-count neural recording, focal cortical stimulation, and accurate neural decoding, as required of bidirectional brain-computer interfaces. The disclosed systems are configured to better decode and encode neural signals, and to expand the population of patients who could benefit from neural interface technology.

Embodiments of the present disclosure include a minimally invasive method for implanting a neural interface including forming a cranial incision in the skull, where the cranial incision has an entry angle approximately tangential to the cortical surface, incising the dura, engaging an insertion paddle with a pocket of the neural interface, advancing the neural interface through the cranial incision into a target region by advancing the insertion paddle, where the target region is within the subdural space, positioning the neural interface at the target region, where the neural interface is configured to at least one of record or stimulate the target region, and withdrawing the insertion paddle from the pocket of the neural interface. Optionally, forming the cranial incision includes using a customized oscillating blade. Optionally, incising the dura comprises coagulating and cutting the dura. The method may also include advancing an endoscope through the cranial incision. In some embodiments the cranial incision is between about 300-500 microns in width and several millimeters (up to approximately 2 cm) length. In some embodiments the endoscope is between about 300-400 microns in diameter. In some embodiments positioning the neural interface includes adjusting the placement, depth, and angulation of the neural interface. Additionally, positioning the neural interface is guided by fluoroscopy or other imaging modalities including, but not limited to, computed tomography (CT), magnetic resonance imaging (MRI), or ultrasound.

In some embodiments a method for attaching a plurality of microelectrode arrays into a modular assembly includes aligning proximal holes of a first microelectrode array from the plurality of microelectrode arrays to distal holes of a second microelectrode array from the plurality of microelectrode arrays, applying a cyanoacrylate to overlapping regions of the aligned proximal and distal holes, and curing the overlapping regions by exposing the overlapping regions of the aligned proximal and distal holes to ultraviolet light. In some embodiments such modular assemblies can be configured to comprise 1,058-4,096 electrodes or more, potentially for simultaneous insertion.

In some embodiments a method for fabricating a neural interface includes fabricating one or more microelectrode arrays, fabricating a pocket, aligning the fabricated pocket with one or more alignment holes of the distal end of a microelectrode array of the one or more microelectrode arrays, and applying a pressure to attach the fabricated pocket to the microelectrode array of the one or more microelectrode arrays. Further, the fabrication of the pocket may include the steps of laser-cutting a pocket area from adhesive-backed polyimide film, wherein the pocket area is sized to include both a distal end of the microelectrode array and a rectangular appendage, and folding the rectangular appendage under an adhesive side of the adhesive-backed polyimide film to create an inner pocket.

In some embodiments a neural interface for at least one of recording or stimulating brain areas may include a plurality of microelectrode arrays, wherein each of the plurality of micro electrode arrays is connected to each other, at least one connector ribbon comprising one or more connector traces from each of the plurality of microelectrode arrays, and a pocket configured to receive an insertion paddle, wherein the pocket is formed on an opposing side of a recording or stimulating surface of the plurality of microelectrode arrays, wherein the neural interface is configured to at least one of record or stimulate brain areas.

In some embodiments a method of using a neural interface in a subject with a condition, the method comprising: implanting the neural interface against a brain of the subject, the neural interface comprising: a flexible substrate, a plurality of microelectrode arrays disposed on a flexible substrate and arranged in a plurality of modules that are removably connected together, the plurality of microelectrode arrays defining a neural interface surface, each of the plurality of modules mechanically connected to each other, each of the plurality of microelectrode arrays comprises electrodes that do not penetrate the surface of the brain against which the electrodes are positioned, and a pocket that receives an insertion paddle and is formed on an opposing side of the neural interface surface; recording, using the implanted neural interface, neural signals from the brain; decoding the recorded neural signals; and controlling a secondary device in accordance with the decoded neural signals.

In some embodiments a method of using a neural interface in a subject with a condition, the method comprising: implanting the neural interface against a brain of the subject, the neural interface comprising: a flexible substrate, a plurality of microelectrode arrays disposed on a flexible substrate and arranged in a plurality of modules that are removably connected together, the plurality of microelectrode arrays defining a neural interface surface, each of the plurality of modules mechanically connected to each other, each of the plurality of microelectrode arrays comprises electrodes that do not penetrate the surface of the brain against which the electrodes are positioned, and a pocket that receives an insertion paddle and is formed on an opposing side of the neural interface surface; recording, using the implanted neural interface, neural signals from the brain; decoding the recorded neural signals; and simulating, using the plurality of plurality of microelectrode arrays, the brain in accordance with the decoded neural signals.

In some embodiments a method of using a neural interface in a subject with a condition, the method comprising: implanting the neural interface against a brain of the subject, the neural interface comprising: a flexible substrate, a plurality of microelectrode arrays disposed on a flexible substrate and arranged in a plurality of modules that are removably connected together, the plurality of microelectrode arrays defining a neural interface surface, each of the plurality of modules mechanically connected to each other, each of the plurality of microelectrode arrays comprises electrodes that do not penetrate the surface of the brain against which the electrodes are positioned, and a pocket that receives an insertion paddle and is formed on an opposing side of the neural interface surface; simulating, using the plurality of plurality of microelectrode arrays, the brain.

### BRIEF DESCRIPTION OF THE DRAWINGS

FIG. 1A depicts a system for a neural interface, in accordance with embodiments of the present disclosure.

FIG. 1B depicts a system for a neural interface, in accordance with embodiments of the present disclosure.

FIG. 2A depicts a microelectrode array for a neural interface, in accordance with embodiments of the present disclosure.

FIG. 2B depicts a neural interface composed of a plurality of microelectrode arrays, in accordance with embodiments of the present disclosure.

FIG. 2C depicts a portion of a microelectrode array, in accordance with embodiments of the present disclosure.

FIG. 2D depicts performance of a microelectrode array, in accordance with embodiments of the present disclosure.

FIG. 2E depicts performance of a microelectrode array, in accordance with embodiments of the present disclosure.

FIG. 2F depicts performance of a microelectrode array, in accordance with embodiments of the present disclosure.

FIG. 3A depicts a microelectrode array for a neural interface, in accordance with embodiments of the present disclosure.

FIG. 3B depicts a portion of a microelectrode array for a neural interface, in accordance with embodiments of the present disclosure.

FIG. 3C depicts a portion of a microelectrode array for a neural interface, in accordance with embodiments of the present disclosure.

FIG. 3D depicts a portion of a microelectrode array for a neural interface, in accordance with embodiments of the present disclosure.

FIG. 4A provides an exploded view for a stage structure for a neural interface, in accordance with embodiments of the present disclosure.

FIG. 4B provides a second view for a stage structure for a neural interface, in accordance with embodiments of the present disclosure.

FIG. 5A illustrates a first step of a delivery system for a neural interface, in accordance with embodiments of the present disclosure.

FIG. 5B illustrates a second step of a delivery system for a neural interface, in accordance with embodiments of the present disclosure.

FIG. 5C illustrates a third step of a delivery system for a neural interface, in accordance with embodiments of the present disclosure.

FIG. 6A illustrates a first step of a delivery system for a neural interface, in accordance with embodiments of the present disclosure.

FIG. 6B illustrates a second step of a delivery system for a neural interface, in accordance with embodiments of the present disclosure.

FIG. 6C illustrates a third step of a delivery system for a neural interface, in accordance with embodiments of the present disclosure.

FIG. 6D illustrates additional views of the steps show in FIGS. 6A-C, in accordance with embodiments of the present disclosure.

FIG. 7A illustrates a delivery system for a neural interface, in accordance with embodiments of the present disclosure.

FIG. 7B illustrates a delivery system for a neural interface, in accordance with embodiments of the present disclosure.

FIG. 7C illustrates a delivery system for a neural interface, in accordance with embodiments of the present disclosure.

FIG. 7D illustrates a delivery system for a neural interface, in accordance with embodiments of the present disclosure.

FIG. 7E illustrates a delivery system for a neural interface, in accordance with embodiments of the present disclosure.

FIG. 7F illustrates a delivery system for a neural interface, in accordance with embodiments of the present disclosure.

FIG. 7G illustrates a delivery system for a neural interface, in accordance with embodiments of the present disclosure.

FIG. 8 provides an implanted view of a neural interface, in accordance with embodiments of the present disclosure.

FIG. 9A illustrates experimental data, including no stimulation, in accordance with embodiments of the present disclosure.

FIG. 9B illustrates experimental data, including stimulation of the snout, in accordance with embodiments of the present disclosure.

FIG. 9C illustrates experimental data, including stimulation of the hindlimb, in accordance with embodiments of the present disclosure.

FIG. 9D illustrates experimental data from stimulation, in accordance with embodiments of the present disclosure.

FIG. 9E illustrates experimental data from stimulation, in accordance with embodiments of the present disclosure.

FIG. 9F illustrates experimental data from recording, in accordance with embodiments of the present disclosure.

FIG. 9G illustrates experimental data from stimulation, in accordance with embodiments of the present disclosure.

FIG. 10A illustrates experimental data from recording, in accordance with embodiments of the present disclosure.

FIG. 10B illustrates experimental data from recording, in accordance with embodiments of the present disclosure.

FIG. 10C illustrates experimental data from recording, in accordance with embodiments of the present disclosure.

FIG. 10D illustrates experimental data from recording, in accordance with embodiments of the present disclosure.

FIG. 10E illustrates experimental data from recording, in accordance with embodiments of the present disclosure.

FIG. 10F illustrates experimental data from recording, in accordance with embodiments of the present disclosure.

FIG. 11A illustrates experimental data from decoding, in accordance with embodiments of the present disclosure.

FIG. 11B illustrates experimental data from decoding, in accordance with embodiments of the present disclosure.

FIG. 11C illustrates experimental data from decoding, in accordance with embodiments of the present disclosure.

FIG. 11D illustrates experimental data from decoding, in accordance with embodiments of the present disclosure.

FIG. 12A illustrates experimental data from stimulation, in accordance with embodiments of the present disclosure.

FIG. 12B illustrates experimental data from stimulation, in accordance with embodiments of the present disclosure.

FIG. 12C illustrates experimental data from stimulation, in accordance with embodiments of the present disclosure.

FIG. 12D illustrates experimental data from stimulation, in accordance with embodiments of the present disclosure.

FIG. 13A illustrates tactile stimulation locations on the rostrum of the Göttingen minipig used for sensory discrimination and neural decoding, in accordance with embodiments of the present disclosure.

FIG. 13B illustrates placement of two electrode arrays on the cortical surface overlying the rostrum somatosensory cortex, in accordance with embodiments of the present disclosure.

FIG. 13C illustrates baseline architecture of the convolutional neural network used for decoding stimulated location, in accordance with embodiments of the present disclosure.

FIG. 13D illustrates a complete grid of electrodes recruited for decoding, in accordance with embodiments of the present disclosure.

FIG. 13E illustrates a sparse grid of electrodes recruited for decoding, in accordance with embodiments of the present disclosure.

FIG. 13F illustrates a dense grid of electrodes recruited for decoding, in accordance with embodiments of the present disclosure.

FIG. 13G illustrates a confusion matrix for neural decoding using the complete grid, in accordance with embodiments of the present disclosure.

FIG. 13H illustrates a confusion matrix for neural decoding using the sparse grid, in accordance with embodiments of the present disclosure.

FIG. 13I illustrates a confusion matrix for neural decoding using the dense grid, in accordance with embodiments of the present disclosure.

FIG. 13J illustrates decoding accuracies for each tactile stimulation location and control, in accordance with embodiments of the present disclosure.

FIG. 13K illustrates a schematic showing bilateral placement of 1024-channel electrode arrays for motor decoding during volitional walking, in accordance with embodiments of the present disclosure.

FIG. 14A illustrates a stimulation waveform used for in vitro confirmation of safe polarization potential, in accordance with embodiments of the present disclosure.

FIG. 14B illustrates example traces for an electrode near the stimulation electrode for 8 stimulation trial recordings, in accordance with embodiments of the present disclosure.

FIG. 14C illustrates traces corresponding to the traces shown in FIG. 14B when the animal is under heavier anesthesia, in accordance with embodiments of the present disclosure.

FIG. 14D illustrates traces corresponding to the traces shown in FIG. 14B without stimulation under light anesthesia, in accordance with embodiments of the present disclosure.

FIG. 14E illustrates stimulated activity plotted against control activity under weak anesthesia, in accordance with embodiments of the present disclosure.

FIG. 14F illustrates stimulated activity plotted against control activity under strong anesthesia, in accordance with embodiments of the present disclosure.

FIG. 14G illustrates activity across the two adjacently placed arrays with stimulation applied at the highlighted electrode, in accordance with embodiments of the present disclosure.

FIG. 14H illustrates activity across the array without stimulation, using the same color scale as in FIG. 14G, in accordance with embodiments of the present disclosure.

FIG. 14I illustrates differential activity across the arrays, calculated as the difference between FIGS. 14G and H, in accordance with embodiments of the present disclosure.

FIG. 14J illustrates a map of differential stimulated activity between light and heavy anesthesia, in accordance with embodiments of the present disclosure.

FIG. 15 illustrates an electrode array comprising more than 4,000 channels, in accordance with embodiments of the present disclosure.

FIG. 16 illustrates an electrode array on the cortical surface following a small frontal craniotomy in a Göttingen minipig and the same region of cortical surface is shown immediately following array removal, in accordance with embodiments of the present disclosure.

### DETAILED DESCRIPTION

Embodiments of the present disclosure are directed towards a modular and highly scalable brain-computer interface platform that is capable of rapid, minimally invasive surgical deployment over multiple large areas of the cortical surface in a reversible manner, without damaging brain tissue. Embodiments of the system provide high channel counts and high spatial densities of electrode coverage, as well as bidirectional functionality (both neural recording and neural stimulation). The brain-computer interface platform may include one or more thin-film microelectrode arrays configured to conform to the shape of the cortical surface.

In some embodiments, the microelectrode arrays may be designed to be inserted into the subdural space through 400-micron-wide skull incisions made using a precision oscillating blade, guided by real-time imaging and fiberoptic endoscopy, at a rate of more than 1,000 electrodes per minute. The disclosed brain-computer interface platform may facilitate human clinical use of brain-computer interface technology by delivering the microelectrode numbers and spatial densities required for advanced, high-performance brain-computer interface applications, in a safe and time-efficient manner that is compatible with proven and reliable neurosurgical techniques.

The disclosed systems may provide the ability to implant large numbers of microelectrodes at high spatial densities, without damaging the brain, in a manner that is potentially reversible and that can be performed rapidly, relatively painlessly, and eventually without the need for general anesthesia.

The disclosed systems may be used for neural recording and stimulation of anatomic and functional targets throughout the human brain. The disclosed systems may provide improved scalability as opposed to conventional systems.

Scalability is a particularly important factor in designing neural interfaces with the intent to reach the large patient populations who stand to benefit from brain-computer interface technology. In considering applications such as paralysis, visual impairment, epilepsy, and some forms of cognitive impairment, the number of potential patients in the United States alone numbers in the many millions. Thus, it is important that the morbidity of the insertion procedure and the amount of normal brain tissue damaged or disrupted should be minimized and should not substantially increase as the number and density of implanted electrodes increase. Similarly, the total time required for implantation should scale in a favorable manner with respect to the number of electrodes. While conventional systems for brain-computer interfaces support increasing channel count by orders of magnitude, it is not practical to lengthen surgical times by the same factors as doing so would mean prolonging a typical 1-hour operation to 10 hours or 100 hours or even longer. Thus, the disclosed systems illustrate an electrode array design and minimally invasive insertion technique that permit thousands of microelectrodes to be positioned on the cortical surface in a manner that scales favorably in terms of safety, electrode number and density, the spatial extent of the neural interface, and insertion time. Embodiments of the present disclosure may use thin-film surface microarrays configured to reduce neural tissue damage to undetectable levels. Thus, large areas of the cortical surface can be covered by these arrays without any appreciable damage to underlying brain tissue. For example, the effective area of the microelectrode arrays described herein is 0.48 cm² per 529-channel module or 1.56 cm² per 1024-channel module. Further, in some embodiments the arrays can be connected in a modular fashion and inserted simultaneously, at an effective rate exceeding 40 s per cm² of cortical surface. Embodiments of the present disclosure may also be used for concurrent implantation over multiple functional areas of the cortical surface in both hemispheres. The disclosed embodiments illustrate that a thin-film-based neural interface may be positioned over the majority of accessible human neocortex.

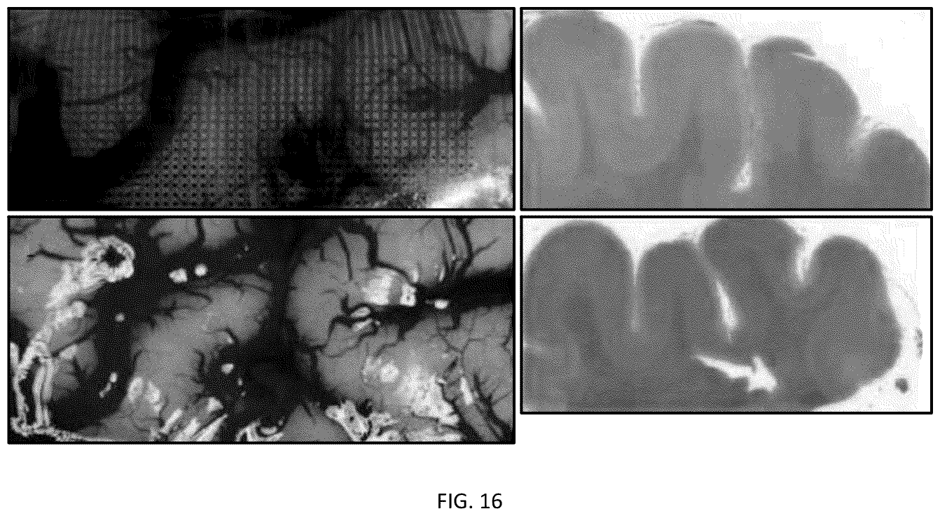

Reversibility, in the sense of having the potential for explanting or replacement of the neural interface without damage to surrounding neural tissue, is another important factor in designing neural interfaces. Any implant may be expected to induce a certain degree of tissue encapsulation, but the anticipated tissue damage associated with removing multiple conventional penetrating microelectrodes is greater than that associated with removing and replacing the cortical surface arrays described herein. It has been proven experimentally that placement and manipulation of the arrays described here causes no macroscopically or microscopically detectable changes in the brain in the short-term (immediate) or long-term (30 days) time frames, as shown in FIG. 16, for example, and described in greater detail below.

The neural interfaces described herein may include microelectrode arrays that are less invasive, more easily inserted and removed without damage to normal brain tissue, and potentially more stable in the long-term. Additionally, with neural decoding techniques they may be capable of high temporal- and spatial-resolution recordings for effective use in in vivo applications, including brain-computer interfaces. For example, neural decoding techniques may utilize surface array techniques to correlate neural activity with motor function for the control of neural prostheses in physically impaired patients. Further, high-density electroencephalography may be used in decoding neural representations of speech, enabling the articulation of words and short sentences, which can be used to allow individuals with conditions such as aphasia to communicate. These applications require the implanted arrays to be reliable and chronically stable. As a result, ultra-flexible arrays, such as those described herein, may provide a promising alternative to more traditional ECoG grids, as they offer a more stable biotic-abiotic interface and in turn promote improved long-term performance. Recently developed thin-film μECoG arrays, such as those described herein, have demonstrated the ability to conform to the brain surface safely for several months while reliably recording multi-unit and single-unit activity in certain anatomic settings. These arrays can also serve as stimulation and biosensing platforms for multi-modal and closed-loop brain-computer interface applications. Further, the disclosed systems provide a novel, minimally invasive approach to reliably implanting high-density arrays, while not requiring the extensive brain surface exposure typically required of higher channel counts in conventional systems.

The disclosed system includes a neural interface, capable of functioning as a brain-computer interface, including the ability to perform the key functions of neural recording, neural decoding, and cortical stimulation. Further, the disclosed neural interface may include one or more microelectrode arrays. Still further, each of the microelectrode arrays may incorporate electrodes of different sizes on the same array, which may in turn facilitate the key functions of recording, decoding and stimulation.

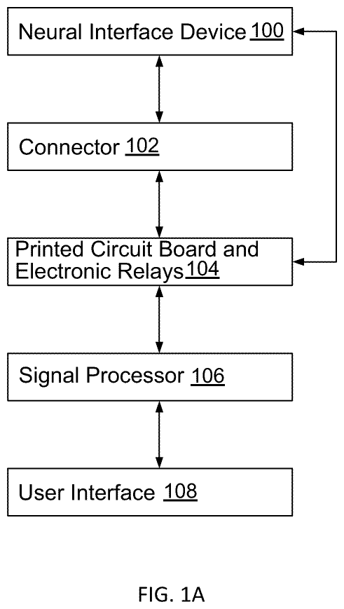

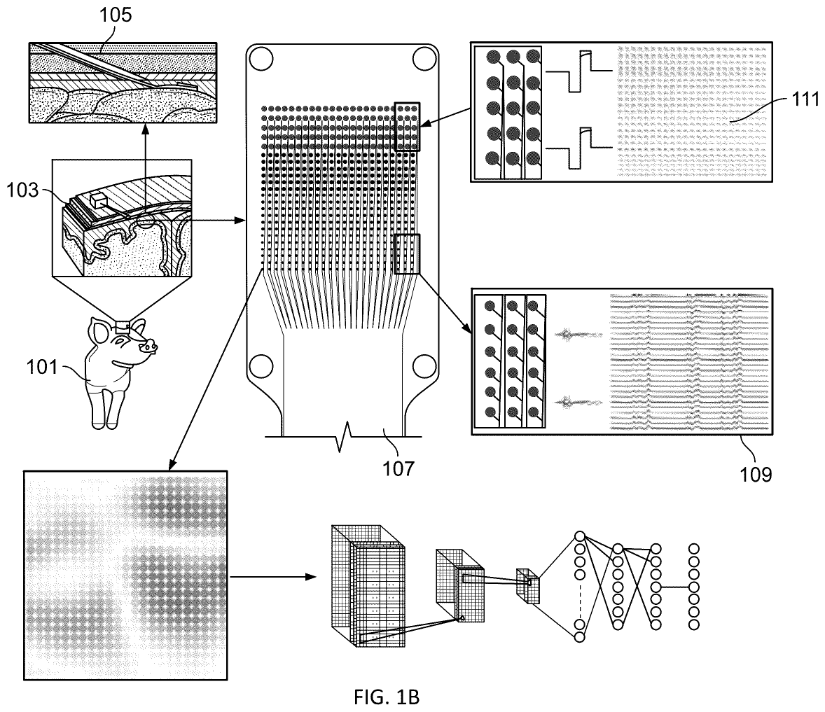

FIG. 1A depicts a system for a neural interface, in accordance with embodiments of the present disclosure. FIG. 1A provides a schematic diagram of a neural interface system. As illustrated, a neural interface device 100 including electrode arrays capable of recording or stimulating may be implanted within the brain. A cable may connect the neural interface device to a connector 102 located outside of the skull. The cable may interface with a printed circuit board and other electronic signal relays 104. The printed circuit board and electronic signal relays 104 may be configured to provide stimulation or recording commands to the neural interface device 100. A signal processor 106 may be configured to receive and/or output signals to the printed circuit board and electronic signal relays 104. The signal processor 106 may also be configured to generate a display of the recorded or stimulated neural signals on a user interface 108, that may be on a laptop, mobile device, computer or the like. Digital input/output signal processing may occur between the user interface 108 and the signal processor 106 and between the signal processor 106 and the printed circuit board and electronic signal relays 104. Further, power sources may be configured to provide power to all of the elements of the neural interface system including the neural interface device 100, connector 102, printed circuit board and electronic signal relays 104, signal processor 106, and user interface 108. As will be discussed herein, the neural interface device 100 may include a microelectrode array that is connected via a connector 102 to a printed circuit board and related electronic signal relays. Electrophysiology chips may be bonded to the printed circuit board 104 so that they can communicate via digital input output signals and power interfaces to signal processor 106 which may in turn connect to a user interface 108. In some embodiments, signals from the electrophysiology chips may be fed back to the neural interface sensors or effectors in a closed loop or other format.

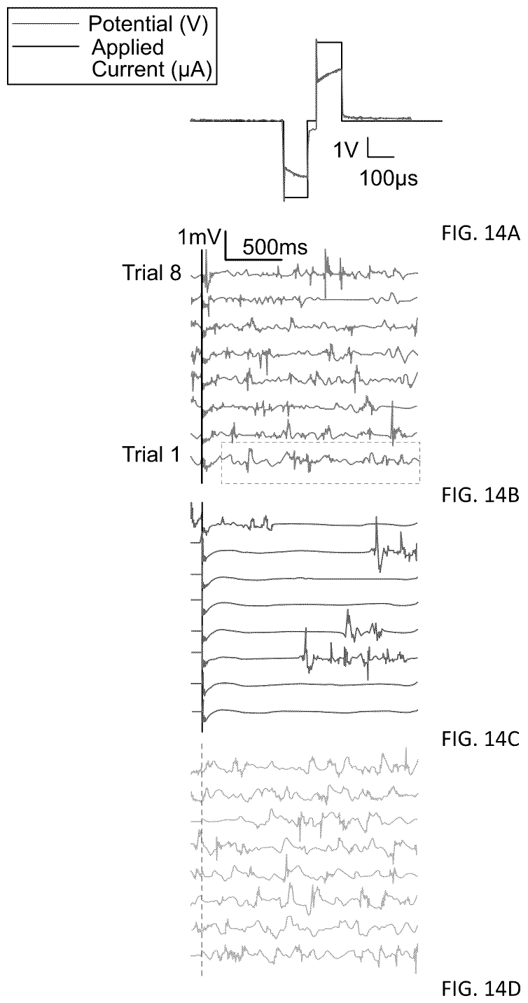

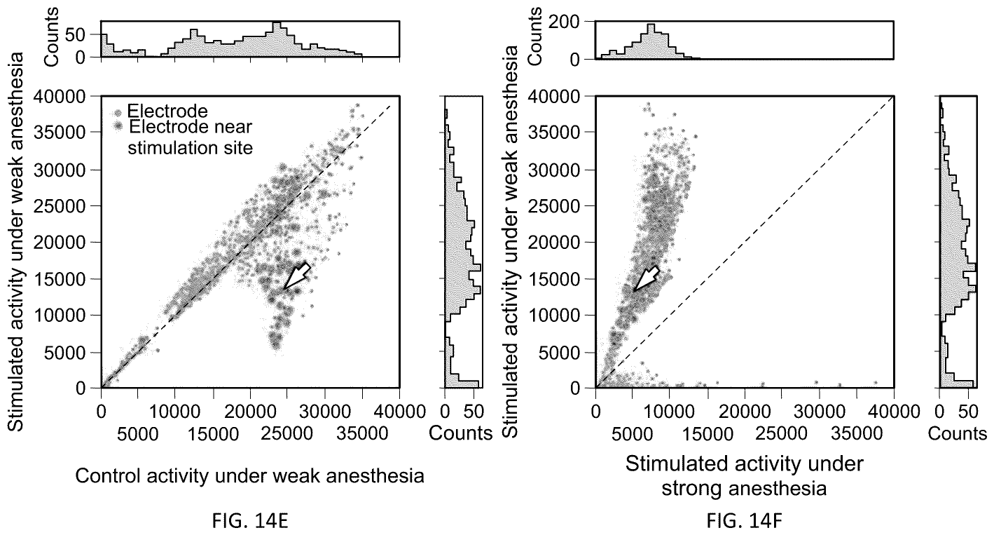

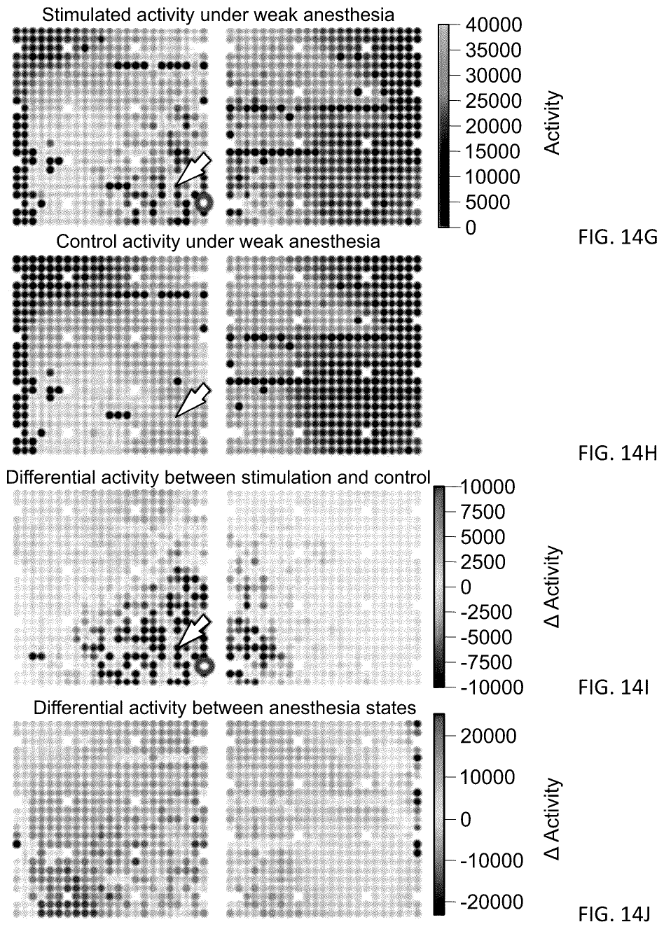

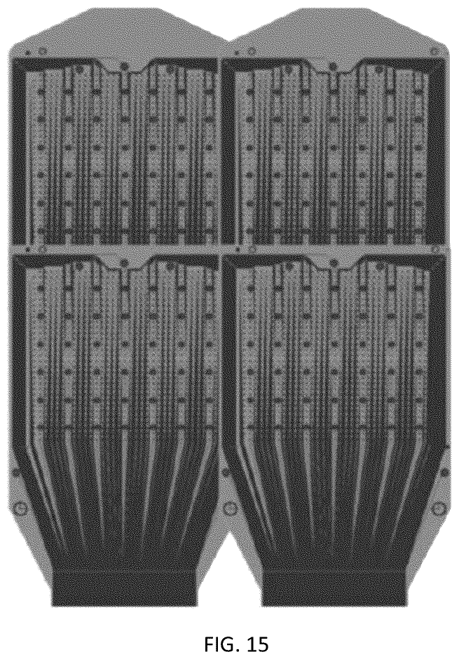

FIG. 1B depicts a system for a neural interface, in accordance with embodiments of the present disclosure. The depicted system illustrates a neural interface including a modular set of thin-film microelectrode arrays which are designed for intradural implantation. As illustrated in FIG. 1B, an animal 101 (e.g., Gottingen minipig, human) may be implanted with a neural interface including a thin-film microelectrode array 107. The neural interface may be implanted using a cranial micro-slit implantation 105 (or via an alternative aperture such as a conventional burr hole or craniotomy) which places the thin-film microelectrode array 107 within the subdural 103 space of the animal brain, with electrodes in direct electrical contact with the cortical surface. In some embodiments, the animal 101 may undergo cranial micro-slit implantation 105 of a set of subdural micro-electrocorticography arrays including a total of over 2000 or more microelectrodes, in modules containing 529 or 1024 channels each. A group of 200 micron microelectrodes and example stimulation waveform traces 111 and resulting post-stimulus activity is recorded over the entire array. A group of 20 micron microelectrodes were shown in detail with traces from recorded neural activity 109. As illustrated, the neural interface may be configured for neural recording and/or stimulation. For example, the neural interface may be configured to record spontaneous neural activity 111, as well as stimulus-evoked neural activity 109. In some embodiments, the thin-film microelectrode array 107 may include more than 2000 microelectrodes, in modules comprising 529 channels each. In an alternative embodiment shown in FIG. 14, the thin-film microelectrode array 107 could include more than 4,000 microelectrodes, in modules comprising 1,024 channels. The microelectrode arrays may be configured to provide multichannel data used in a variety of electrophysiologic paradigms to perform neural recording and neural decoding (using, e.g., neural network architectures as shown in FIG. 1B) of both spontaneous and stimulus-evoked neural activity as well as decoding and focal stimulation of neural activity across a variety of functional brain regions.

In some embodiments, the neural interface includes a modular set of thin-film microelectrode arrays designed for subdural implantation. Each of the microelectrode arrays may be connected to an interposer, which is in turn connected to a customized hardware interface for neural recording and stimulation. In some embodiments, two, four, or any other number of microelectrode arrays may be connected to form a neural interface device. The connector portion of each 529-channel microelectrode array or 1024-channel microelectrode array module may be configured to pass through a dural incision and a cranial micro-slit incision, and tunneled under the scalp as needed, and then connected to an individual head stage. The head stages as illustrated may not be configured for subgaleal implantation and may be designed for acute use only. However, certain head stages or receiving stages may also be configured for subgaleal implantation, chest wall implantation, or long-term implantation within other compartments within the body for long-term (chronic) use (many years). Some embodiments of the present disclosure may contain integrated electronics and wireless functionality incorporated within a fully implantable package that will obviate the need for transcutaneous connection to an external head stage.

Examples of the microelectrode array such as one for the neural interface described herein may be found at least at U.S. Provisional Application No. 63/255,724, entitled “Apparatus, Systems, and Methods for High-Bandwidth Neural Interfaces”, filed on Oct. 14, 2021, which is hereby incorporated by reference.

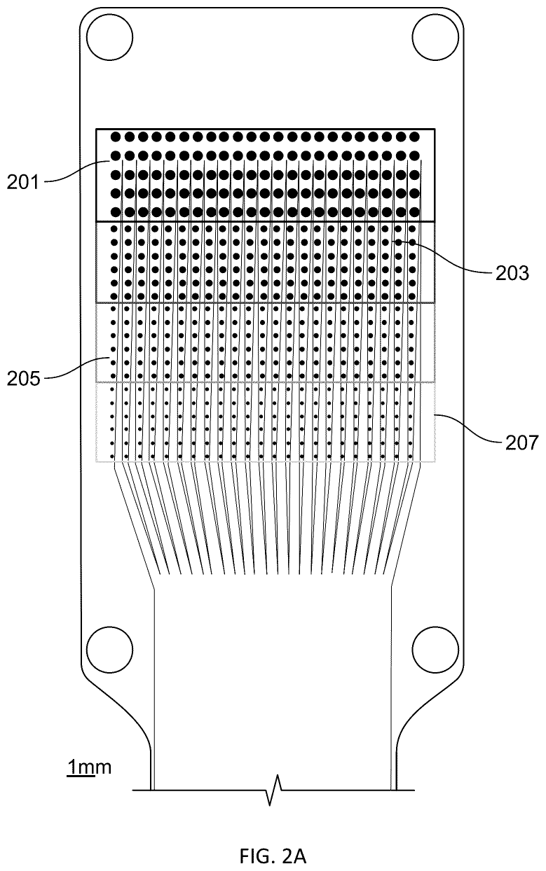

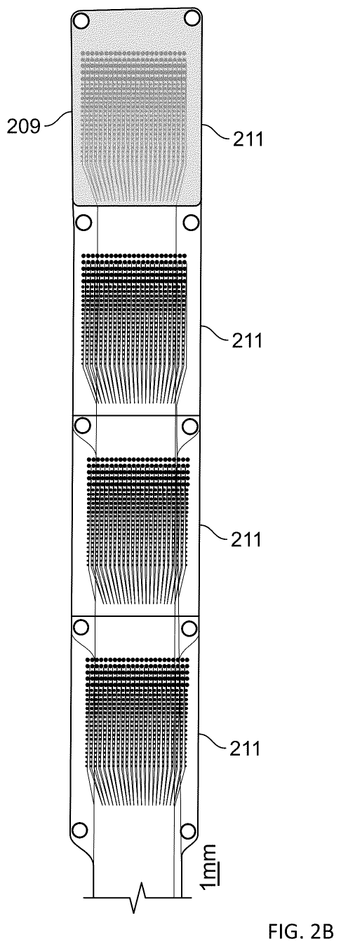

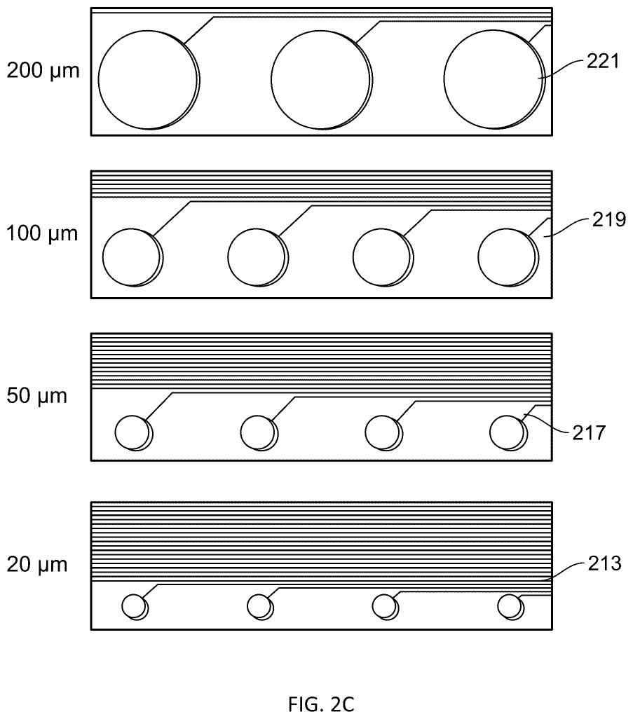

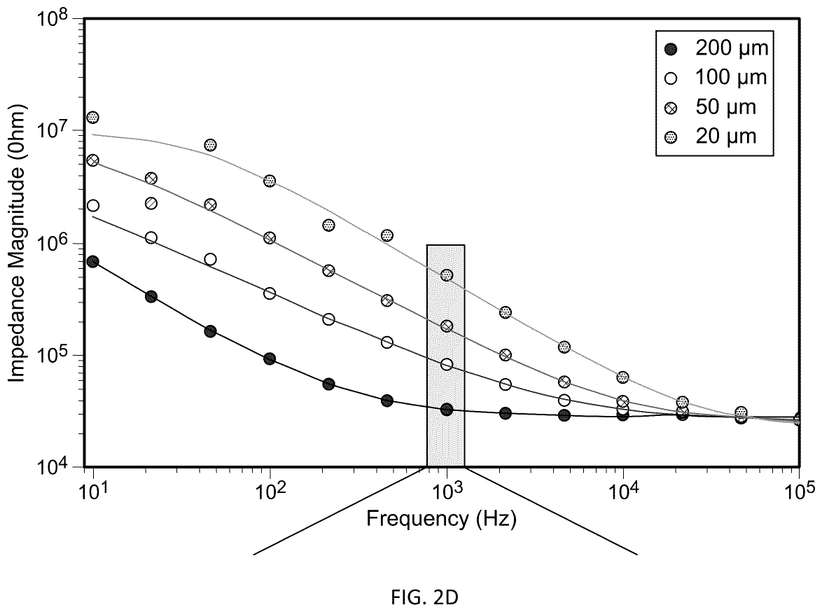

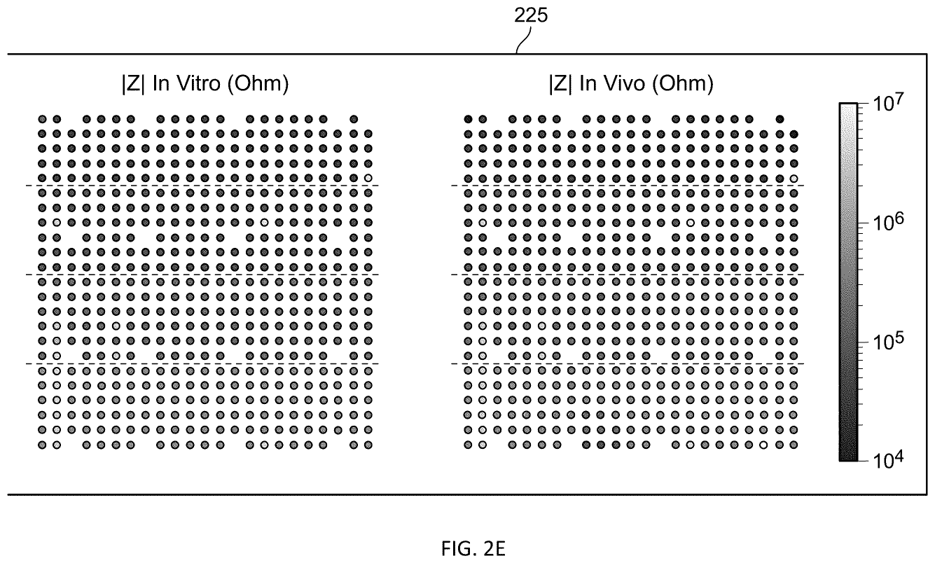

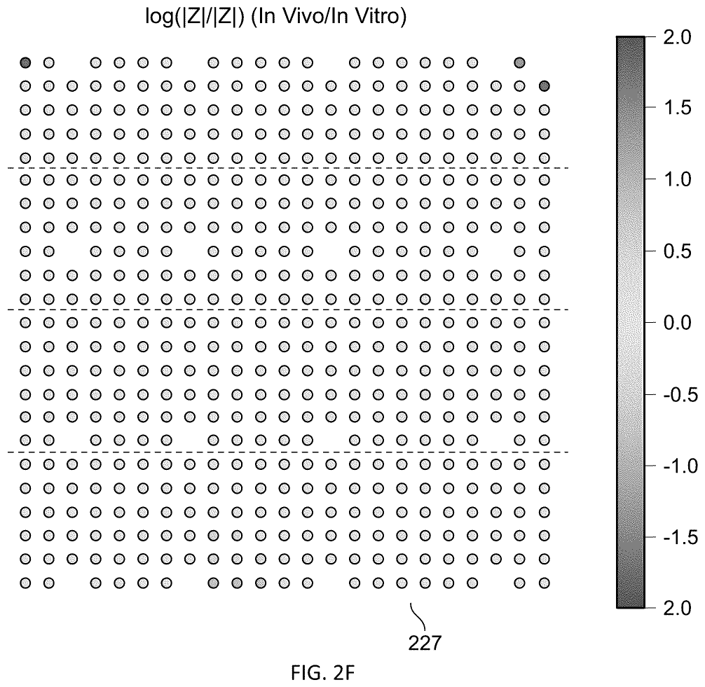

FIG. 2A depicts a microelectrode array for a neural interface, in accordance with embodiments of the present disclosure. In accordance with the embodiments described herein, the microelectrode array may be configured for neural recording, neural decoding, and neural stimulation. The disclosed microelectrode array contains a plurality of electrodes each having a range of sizes. For example, the microelectrode array in FIG. 2A includes 200 micron diameter electrodes 201, 100 micron diameter electrodes 203, 50 micron diameter electrodes 205, and 20 micron diameter electrodes 207.

FIG. 2B depicts a neural interface composed of a plurality of microelectrode arrays, in accordance with embodiments of the present disclosure. Illustrated is a quadruple-connected modular array assembly 209 of four 529-electrode modules 211 with a single pocket, prepared for simultaneous implantation of 2116 electrodes. Alternatively, a double-attached array assembly of two 529-electrode modules with a single pocket, configured for the simultaneous implantation of 1058 microelectrodes, may be used.

In some embodiments, similar to the quadruple-attached array assembly 209, a plurality of microelectrodes may be modularly connected. For example, microelectrode arrays may be configured into a chain of connecting modules to achieve greater functional cortical coverage. Assembly of such configurations may be achieved by carefully aligning the proximal holes of a preceding microelectrode array in a chain to the distal holes of its succeeding array. The arrays may then be bonded by applying a small amount of ISO 10993 biological tested UV-cure cyanoacrylate to the overlapping regions of the two array modules. As will be discussed further in the experimental results and working examples, chains consisting of multiple connecting array modules were tested and validated in both in vitro and in vivo experiments.

As illustrated in FIG. 2B, modular configurations of multiple 529-channel arrays can be constructed. Illustrated is a quadruple-connected assembly of four 529-electrode modules with a single pocket, for simultaneous implantation of 2116 microelectrodes over 1.92 square centimeters of cortical surface area.

FIG. 2C depicts a portion of a microelectrode array, in accordance with embodiments of the present disclosure. Illustrated are photomicrographs of 20 micron diameter electrodes 213, 50 micron diameter electrodes 217, 100 micron diameter electrodes 219, and 200 micron diameter electrodes 221 following array release.

FIG. 2D depicts performance of a microelectrode array, in accordance with embodiments of the present disclosure. Illustrated is the electrochemical impedance spectroscopy 223 for each electrode size, with equivalent circuit fits based on a Randles circuit.

FIG. 2E depicts performance of a microelectrode array, in accordance with embodiments of the present disclosure. Illustrated is the in vitro (left) and in vivo (right) impedance maps 225 of recording channels over a complete 529-channel array, measured at 1 kHz, with microscope images for each electrode size included as references. Stimulation and ground channels are depicted as gaps in the plot.

FIG. 2F depicts performance of a microelectrode array, in accordance with embodiments of the present disclosure. Illustrated is a map of the ratio between in vivo and in vitro impedance at 1 kHz, displaying minimal changes across most of the array following implantation in accordance with the working examples and experimental results described herein.

In some embodiments, the microelectrode arrays illustrated in FIGS. 2A-2F were fabricated on 8″ silicon wafers using a spin-on BPDA-PPD (biphenyltetracarboxylic dianhydride, p-phenylenediamine) polyimide (PI-2611). The fabrication process briefly included spin-coating, soft-bake, and 350° C. vacuum cure of an approximately 10.5 μm layer of polyimide; photolithographic patterning, deposition, and liftoff of 20 nm/210 nm/20 nm Ti/Pt/Ti trace metal; O₂ plasma roughening of the polyimide surface; spin-coating, soft-bake, and 370° C. vacuum cure of an approximately 10.5 μm layer of polyimide; aluminum hard mask deposition and patterning for polyimide outline and electrode site opening; polyimide etch and electrode surface exposure in O₂/CF₄ plasma; hard mask strip; photolithographic patterning, deposition, and liftoff of 20 nm/20 nm/500 nm of Ti/Pt/Au bond pad metallization; and O₂ plasma post-clean of the polyimide surface. Following microfabrication, the devices were released in deionized water, optically inspected for trace, electrode, and pad defects, dehydration baked, and thermocompression bonded to an organic interposer using a flip-chip tool.

In some embodiments, prior to assembly, the bonded microelectrode array-interposer assemblies may be optically inspected in bond, cable, and electrode areas, and a sampling of electrodes may be characterized electrochemically. Electrochemical characterization may be performed on a potentiostat (Wavedriver 100, Pine Research, Durham, North Carolina, United States of America) in a 3-electrode configuration (with Ag/AgCl reference electrode and Pt coil counter electrode), and include cyclic voltammetry (CV) and electrochemical impedance spectroscopy (EIS) on at least one electrode per size in phosphate buffered saline (PBS) at pH 7.4. The CV measurements may be performed (from 0 to 1.2 V to −0.65 V to 0 V relative to the reference electrode) to confirm electrode surface identity using platinum oxidation and Pt—O reduction peaks, hydrogen adsorption, and H₂ oxidative desorption. In addition, CV measurements may provide information on charge storage capacity and real surface area, and identify the water window. EIS measurements may be performed from 10 Hz to 10 kHz (on each electrode size) to confirm that 1 kHz impedance and cutoff frequency are within expected ranges, and to provide references for later in vitro impedance mapping performed using the Intan chips in a two-electrode configuration. In vitro impedance mapping may be performed in PBS on fully assembled devices (across all electrodes) at 100 Hz, 200 Hz, 500 Hz, 1,000 Hz, 2000 Hz, and 5,000 Hz using the Intan chips in our custom 528-channel head stage.

As discussed, prior to assembly, bonded microelectrode array-interposer assemblies may be optically inspected in bond, cable, and electrode areas. Example photographs and photomicrographs are shown in FIG. 2B. Process yield may be evaluated with respect to electrode connectivity, and confirmed to be >93% after optimization, by impedance characterization of 4,224 electrodes from eight arrays. A set of electrodes of each size may also be characterized electrochemically by cyclic voltammetry (CV) and electrochemical impedance spectroscopy (EIS). EIS measurements for each electrode size display typical positive shifts in cutoff frequencies with decreases in electrode size, and impedance magnitude at 1 kHz also follows the expected upward trend with decreasing electrode size. In vivo electrode impedance mapping results displaying electrode yield and impedance uniformity are summarized in FIG. 2E, displayed as impedance amplitude at the 1 kHz cutoff frequency. Electrode impedance demonstrates a predictable dependence on electrode surface area, ranging from an average of 526 kΩ for 20 μm electrodes to 32 kΩ for 200 μm electrodes as measured by a potentiostat (and ranging from an average of 642 kΩ for 20 μm electrodes to 46.9 kΩ for 200 μm electrodes as measured by the Intan electronics). The microelectrode arrays described herein maintains robustness during the implantation process, as illustrated by the plotted ratio of impedance before and after implantation as is illustrated in FIG. 2F, there little impact on the impedance across the array, excepting some electrodes on the distal corners of the array.

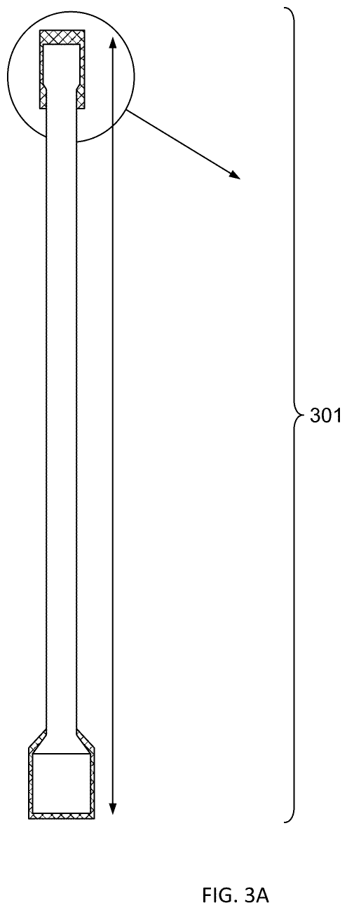

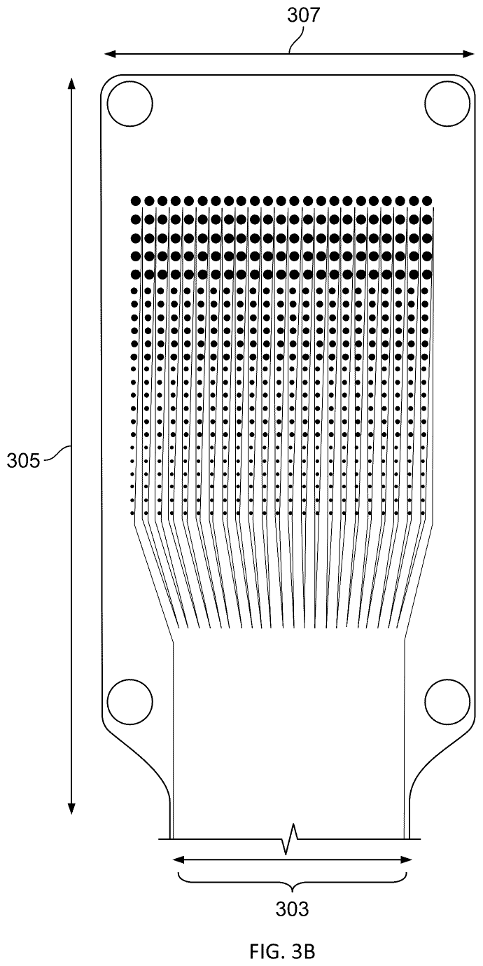

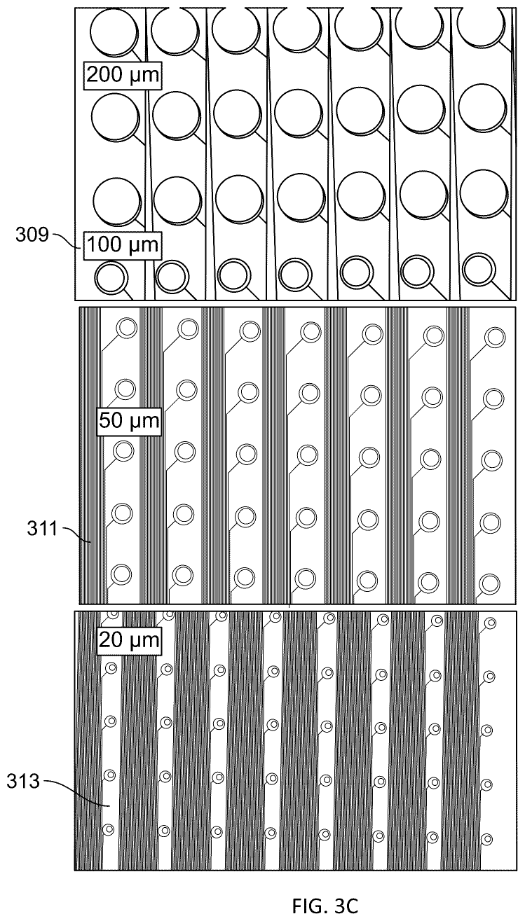

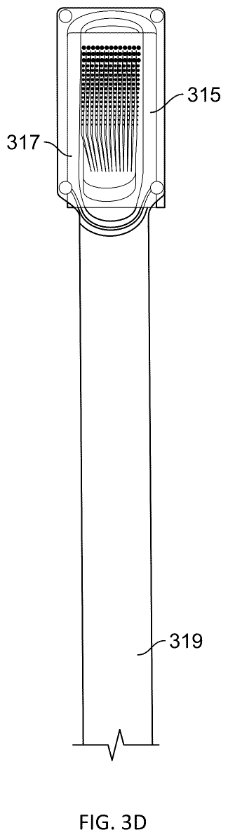

FIG. 3A depicts a neural interface, in accordance with embodiments of the present disclosure. The neural interface of FIG. 3A has a length 301 of approximately 15.5 cm. The neural interface includes a flexible polyimide-based array with 529 electrodes each having a diameter between 20-200 microns and being configured for at least one of recording and/or stimulation. The electrode array can be disposed on a flexible substrate. The neural interface also includes long interconnects for full access to the brain surface via subdural implantation via a minimally invasive “cranial micro-slit insertion” method.

FIG. 3B depicts a portion of a microelectrode array for a neural interface such as the one illustrated in FIG. 3A, in accordance with embodiments of the present disclosure. The electrical array may include a first end having a width 307 of approximately 8.5 millimeters. A second end has a width 303 of approximately 5.5 millimeters and may be configured to connect the electrode array with the interconnects. The length of the microelectrode array 305 may be approximately 11.6 millimeters.

FIG. 3C depicts a portion of a microelectrode array for a neural interface, in accordance with embodiments of the present disclosure. The portion illustrated in FIG. 3C may be a portion of the area depicted in FIG. 3B. As illustrated a single electrode array may include regions containing electrodes having various sizes. For example, a first region 309 may include electrodes having a diameter of 200 microns or 100 microns. A second region 311 may include electrodes having a diameter of 50 microns. A third region 313 may include electrodes having a diameter of 20 microns.

FIG. 3D depicts a portion of a microelectrode array for a neural interface, in accordance with embodiments of the present disclosure. As will be discussed in further detail below. The microelectrode array may be configured to be contained within a pocket configured to be delivered to the target region. For example, a microelectrode array 315 having interconnects 319 may be contained within a pocket 217 for delivery to the target area.

Array pockets 217 may be fabricated as follows. The array pockets may be laser cut from 25 μm thick silicone adhesive-backed polyimide film using a low-power 3 W UV laser. The shape of the planar laser cut pattern may be designed to coincide with the contour of the distal end of the microelectrode array with the addition of a 5.5 mm×11.5 mm rectangular appendage protruding from the proximal edge. The rectangular appendage may be folded under the adhesive side of the laser cut polyimide to occlude the adhesive and create the internal pocket feature of 5.5 mm width and 11.5 mm in length. The dimensions of the internal pocket feature may be chosen to maintain a consistent lap joint width of 1.5 mm between the pocket and microelectrode array. Once the pocket is laser cut and folded into its final configuration, the pocket may then be carefully aligned with 800 μm alignment holes at the distal tip of the microelectrode array and gently compressed using rubber tipped vacuum tweezers. The integrity of the adhesive bond may be evaluated by applying a surgical stylet into the pocket at a force much greater than the true insertion force implemented in a real procedure.

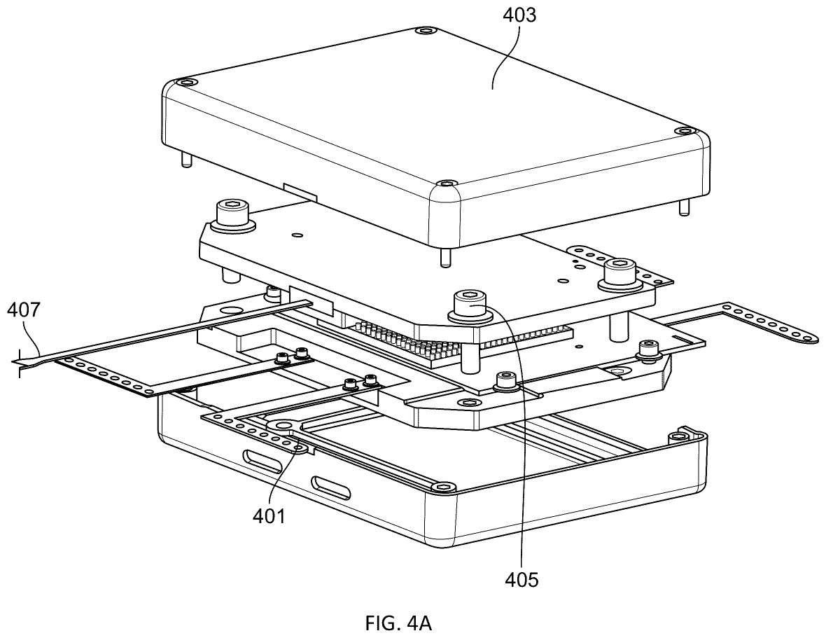

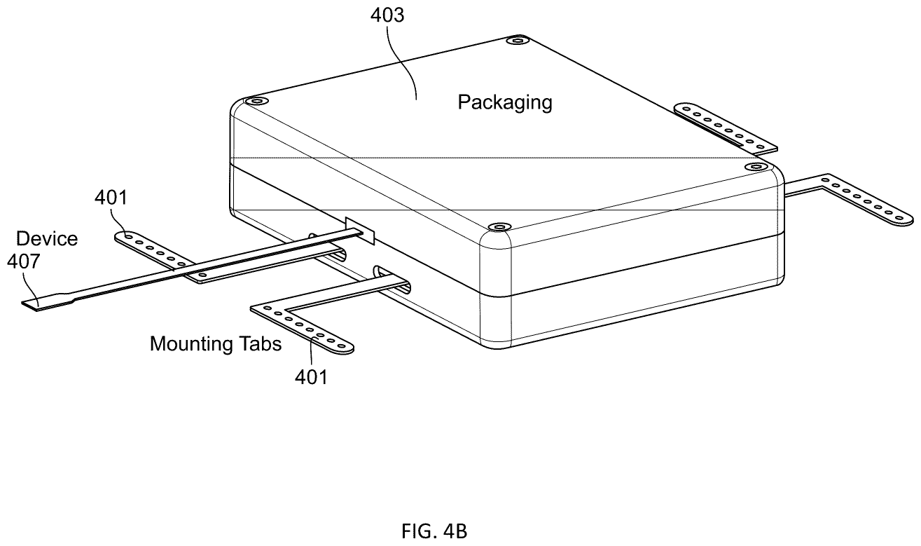

FIG. 4A provides an exploded view for a stage structure for a neural interface, in accordance with embodiments of the present disclosure. As illustrated, the neural interface may form a flexible device 407 that can be connected to a printed circuit board via an interposer. The electronic circuit 405 may be located within and protected by an external head stage and package 403. Additionally, mounting tabs 401 may be used to anchor the system to the skull during a procedure.

In some embodiments, the electronics package may be located outside of the head. In some embodiments, the electronics may implanted between the scalp and the skull such that connected electrodes are positioned on the brain surface but powered electronics are implanted between the skull and scalp, not in contact with the brain. Alternatively, in some embodiments, all of the electronic components may be located within the skull.

FIG. 4B provides a second view for a stage structure for a neural interface, in accordance with embodiments of the present disclosure. Illustrated is the external packaging 403, neural interface device 407, and mounting tabs 401.

FIGS. 4A and 4B may include a customized neural recording and stimulation system based on chips and controllers made, for example, by Intan Technologies (Los Angeles, California, United States of America). The custom amplifier printed circuit boards (PCBs) may be used to interface with the implanted electrode arrays, which each contain eight of the RHD2164 64-channel amplifier chips and one of the RHS2116 16-channel stimulator/amplifier chips, allowing for simultaneous recording from up to 528 channels and stimulation from up to 16 channels. In addition, each board may allow for a hardware reference from one of 16 sites distributed across the array. The digitized data may be transferred from the amplifier boards to an associated Intan Technologies 1,024-channel RHD controller or 126-channel RHS controller using low-voltage differential signaling (LVDS), where it may be stored on a USB-connected computer.

The amplifier boards may be designed to allow each board to be easily coupled to any array-interposer assembly through the inclusion of an array of pogo pins that make contact with an associated pad on the array-interposer assembly, connecting each electrode site with an amplifier input. These two boards may be aligned and held together by two plates with integrated alignment features placed on the outward-facing sides of the boards and screwed together. Additional protection of these electronics may be provided by a custom, 3D-printed casing with strain-relief features for the electrode array and optional mounting braces to fix the entire assembly to the skull.

In some embodiments, the recording computers may interface with either controller via a custom configuration of the Intan Technologies RHX Data Acquisition Software, which may allow for real-time event-triggered averaging in addition to base functionality. The sampling rate for recording is set at 20 kHz per channel, generating data at a rate over 2.5 GB per minute for each set of 1,024 channels. A 60 Hz notch filter may be applied online during recording. For post-hoc analysis of local field potentials, unless otherwise specified, data may be first downsampled to 5 kHz using a Fourier method, and then processed with a 5th-order Butterworth low-pass filter at 250 Hz.

The components illustrated in FIGS. 4A and 4B may be used for acute embodiments only. Some embodiments of the present disclosure may contain integrated electronics and wireless functionality incorporated within a fully implantable package that will obviate the need for transcutaneous connection to an external head stage.

In some embodiments, the signal processor, recording apparatus, and other components of the electronics package may utilize one or more customizable ASICs. Further, data transmission and power supply may utilize wired or wireless connections.

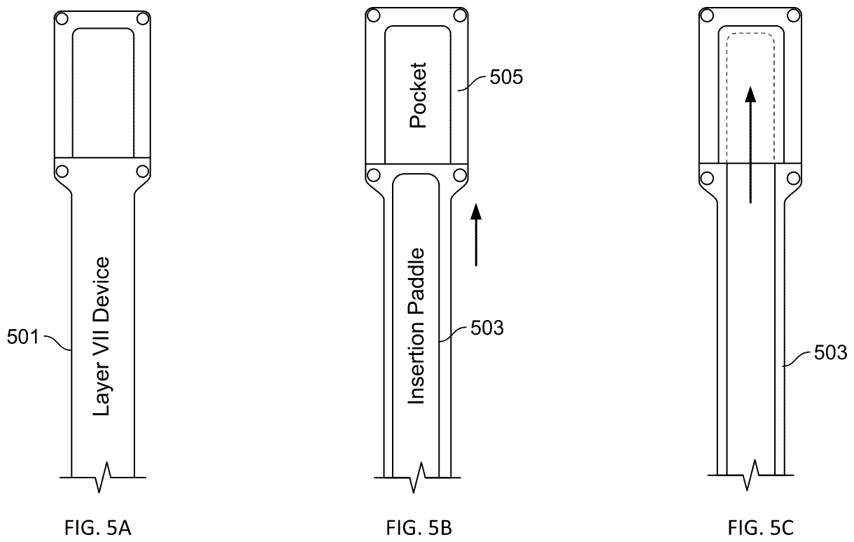

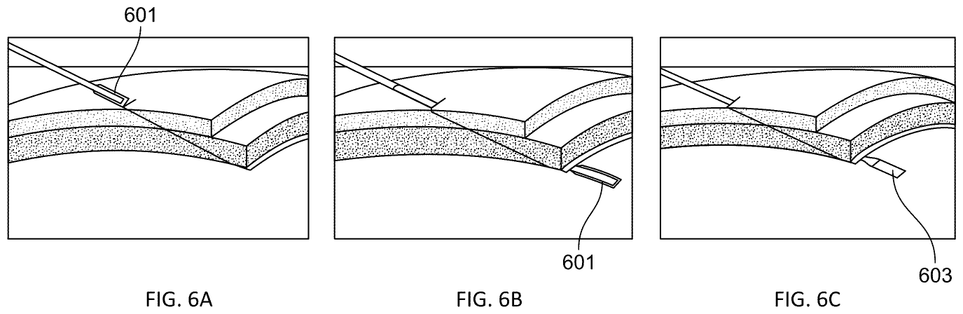

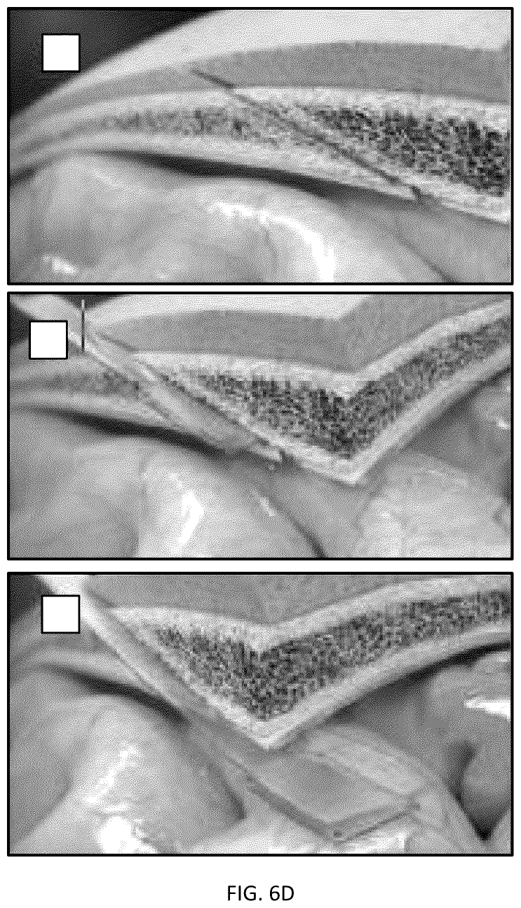

FIGS. 5A-5C and FIGS. 6A-6C illustrate components of a delivery system for a neural interface, namely a “cranial micro-slit insertion” method. The cranial micro-slit insertion method provides a minimally invasive delivery system for the neural interface and includes the steps of making a slit through the skull, incising the dura, and then inserting the flexible device through the slit to the target region with the assistance of an insertion paddle. In some embodiments, the insertion paddle is a semi-rigid insertion paddle that is positioned into the device pocket and used to guide and position the microelectrode array of the neural interface on the surface of the brain.

FIGS. 5A-5C illustrate engagement of the insertion paddle with the neural interface device. FIG. 5A illustrates a first step of a delivery system for a neural interface. As illustrated the neural device 501 may be attached to a pocket 505 sized to receive an insertion paddle 503. FIG. 5B illustrates a second step of a delivery system for a neural interface, where an insertion paddle 503 is aligned with the neural interface device 501. FIG. 5C illustrates a third step of a delivery system for a neural interface, where the paddle 503 is inserted into the pocket 505.

Once the insertion paddle has engaged with the neural interface device as illustrated in FIGS. 5A-5C, the assembly may be inserted through a slit in the skull and past the dura, and positioned adjacent to the target location, upon which the insertion paddle may be withdrawn, leaving the neural interface in place, as is illustrated in FIGS. 6A-6C. FIGS. 6A-6C provide a schematic illustration of the cranial micro-slit insertion method. FIG. 6A illustrates a first step of a delivery system for a neural interface, as the microelectrode array is advanced into the subdural space by a guiding insertion paddle. As illustrated, the insertion paddle is positioned within a pocket of the neural interface device 601. FIG. 6B illustrates a second step of a delivery system for a neural interface, as the microelectrode array is positioned adjacent its target location by the insertion paddle. As illustrated, the insertion paddle is positioned within a pocket of the neural interface device 601. FIG. 6C illustrates how the neural interface, and microelectrode array remains in the subdural space after removal of the insertion paddle or guiding shim. As illustrated, the insertion paddle is retrieved and the neural interface device 603 remains in the subdural space. Additional views of these steps shown in FIGS. 6A-6C are shown in FIG. 6D from an alternative angle.

In connection with the cranial micro-slit insertion method illustrated in FIGS. 5A-6C, a cranial incision (e.g., 400 micron) may be made at an entry angle tangential to the cortical surface. In some embodiments, a customized oscillating blade may be used to make the cranial incision. An endoscope sized to fit (e.g., 350 micron) within the cranial incision may then be inserted through the cranial incision in order to visualize the dura. The dura may then be coagulated and cut under direct endoscopic vision. Endoscopy may also be used to guide the insertion of the electrode array into the subdural space. In some embodiments, an endoscope may be advanced through the cranial incision. In some embodiments, the endoscope may be a fiberoptic endoscope or a flexible endoscope.

In some embodiments, the electrode arrays of the neural interface may be positioned subdurally on the cortical surface under simultaneous endoscopic and fluoroscopic guidance. Manipulation of each thin-film array may be performed using a radiopaque stylet. The stylet tip, may be a guiding shim or an insertion paddle that is designed to fit within a polyimide “pocket” on the reverse side of each array. The placement, depth, and angulation of cranial incisions and electrode arrays may also be guided by fluoroscopy, or by CT- or MRI-based neuronavigation. The guiding shim or insertion paddle may then be removed following fluoroscopic confirmation of array position, leaving only the thin-film subdural microelectrode arrays of the neural interface in position on the cortical surface.

The cranial micro-slit insertion method may be modified using customized sagittal saws including those having novel stabilization methods as well as blade modifications for safety and alignment. Further, the cranial micro-slit insertion method may be used with a computerized navigation guidance system for trajectory planning and alignment. The cranial micro-slit insertion method may involve the use of one or more custom tools, any combination of which may be used in connection with the cranial micro-slit insertion method.

For example, in some embodiments, the cranial incision may be formed by using an oscillating or sagittal saw. In some embodiments the oscillating or sagittal saw device includes a thin blade 400 microns in thickness. The oscillating or saw device may have a length configured to traverse the skull along a trajectory tangential to the cortical surface, for example a length between about 24 mm to 41 mm. The blade may be between 5 mm and 10 mm wide, or any other width configured to keep the procedure minimally invasive. In some embodiments, the device may function based on ultrasound ablation rather than a traditional saw mechanism. Optionally, the blade or saw tool may include a depth stop configured to prevent the tool from damaging the dura or the brain. The blade or saw tool may also include or be designed to function together with a device designed to maintain the trajectory and angle of the saw to remain tangential to the cortical surface in order to ensure optimal placement of the subdural arrays following fashioning the slit.

The disclosed neural interfaces may be used in various applications, including, without limitation, for epilepsy or stroke, or for other conditions in which cortical function is preserved but electrophysiologic interactions between the cortex and other portions of the nervous system and musculoskeletal system are disrupted (as in spinal cord injury or amyotrophic lateral sclerosis). For example, the neural interfaces may be used for strokes and related disorders, by utilizing techniques for cortical stimulation in conjunction with neural recording and decoding in a manner that monitors progress of rehabilitation and provides brain-computer-interface functionality for “assistive technologies” during rehabilitation by interfacing with the preserved or recovering portions of the cortical surface, and possibly modifies stimulation depending on recorded activity. Similarly, the disclosed neural interfaces may be used for assistive or augmentive technologies.

The disclosed system will enable the benefits of high-density, high-channel-count neural interfaces to the millions of patients with neurologic disorders who stand to benefit from this technology.

Additional details regarding various embodiments of neural interfaces can be found in PCT Patent Application No. PCT/US2022/078130, entitled APPARATUS, SYSTEMS, AND METHODS FOR HIGH-BANDWIDTH NEURAL INTERFACES, filed Oct. 14, 2022, which is hereby incorporated by reference herein in its entirety.

### Working Examples and Experimental Results

Embodiments of the disclosed cortical interfaces including the discussed microelectrode arrays and the described slit-insertion method were validated in vivo experiments. Neural interfaces were implanted within Gottingen mini-pigs having substantially similar skull thickness as humans and a well-characterized functional brain anatomy. In vivo experiments demonstrated successful surgical approaches using minimally invasive slit insertion methods, as well as the implantation of highly scalable electrode arrays each having a plurality of electrodes (e.g., 1024 electrodes, 2048 electrodes). In vivo experiments demonstrated the use of multiple arrays per surgery including up to four surface arrays on each hemisphere. As will be discussed below, in vivo experiments demonstrated bidirectional communication of the disclosed cortical interfaces, including both neural recording and stimulation. Neural decoding at a rate exceeding 4 bits per second was demonstrated.

### Experimental Design—Minimally Invasive Surgical Implantation

Neural interfaces built in accordance with the embodiments described herein were implanted within Gottingen mini-pigs using the slit-insertion mechanism described herein.

In vivo testing of the minimally invasive surgical insertion technique and electrode array performance described herein was performed in adult female Göttingen minipigs, selected for well characterized functional neuroanatomy as well as skull thickness comparable to that of adult humans. Local anesthesia was achieved in the region of the skin incisions using intradermal lidocaine. General anesthesia was maintained with isoflurane at levels sufficient to produce analgesia without suppressing electrocorticographic activity, a balance that was facilitated by the minimally invasive nature of the procedure.

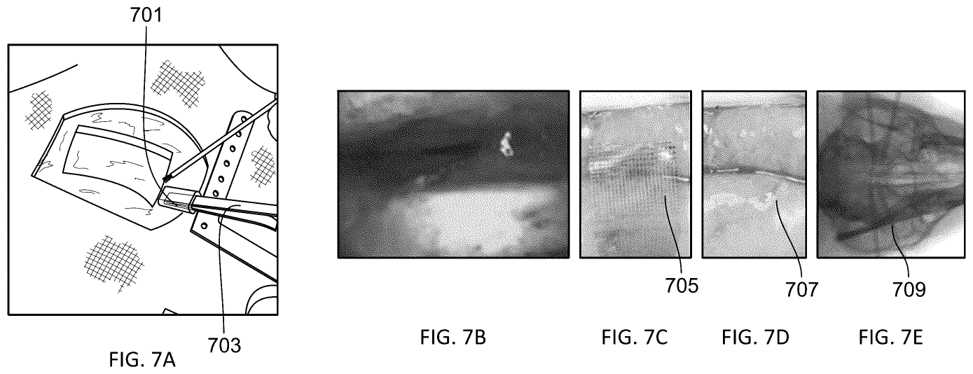

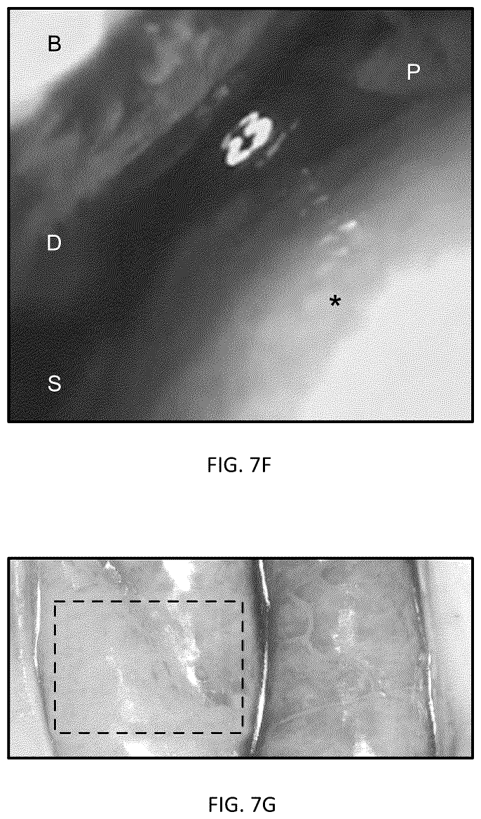

FIGS. 7A-7G provide images from a surgery performed on the Gottingen minipigs and post-mortem, illustrating the slit-insertion method for the neural interface device.

FIG. 7A illustrates a delivery system for a neural interface. In particular, FIG. 7A provides an illustration of the insertion paddle 703 inserting the neural interface device 701 containing the microelectrode array through a cranial slit, as is described above in connection with the cranial micro-slit insertion method.

FIG. 7B illustrates a delivery system for a neural interface, in accordance with embodiments of the present disclosure. In particular, FIG. 7B provides an intraoperative endoscopic view immediately following subdural placement of one thin-film array and shim removal, showing the electrode tail and traces entering the subdural space through a cranial slit.

FIG. 7C illustrates a delivery system for a neural interface, in accordance with embodiments of the present disclosure. A view of the cortical surface postmortem corresponding to the region of subdural array placement, with the array 705 in place is illustrated in FIG. 7C.

FIG. 7D illustrates a delivery system for a neural interface, in accordance with embodiments of the present disclosure. A view of the cortical surface 707 postmortem corresponding to the region of subdural array placement, without the array in place is illustrated in FIG. 7D. As illustrated, there is no appreciable damage to the cortical surface 707.

FIG. 7E illustrates a delivery system for a neural interface, in accordance with embodiments of the present disclosure. In particular, FIG. 7E provides an intraoperative fluoroscopic image (dorsoventral projection) showing four 529-electrode arrays 709 in place, two over each hemisphere.

FIG. 7F provides an intraoperative endoscopic view immediately following subdural placement of one thin-film array, after shim removal, showing the electrode tail and traces entering the subdural space through a cranial slit (B, cut surface of skull seen from within the cranial micro-slit; D, undersurface of the dura; S, subdural space; P, pia of the cortical surface; the asterisk denotes the tail of the array, 5.49 mm in width, passing through the micro-slit.

FIG. 7G provides a view of the intact cortical surface postmortem corresponding to the region of subdural array placement. The shaded outline indicates the position of the array before removal.

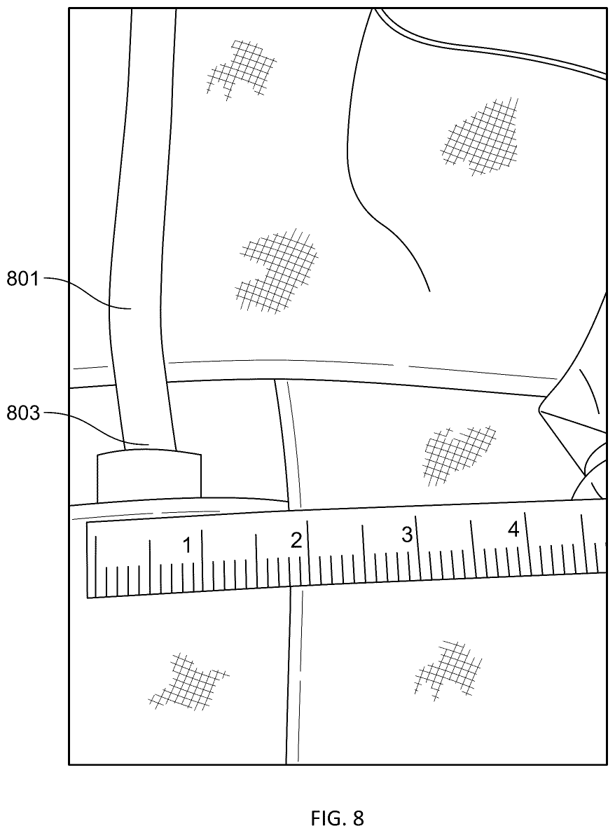

FIG. 8 provides an illustration of the implanted neural interface, illustrating a ribbon 801 that extends from the cranial slit or insertion 803. The ribbon may be configured to include a plurality of connectors that interface with a printed circuit board.

The disclosed working example illustrates the minimal invasiveness and speed of implantation for the disclosed neural interface devices. For example, increasing numbers of cranial micro-slit insertions in a series of three Göttingen minipigs (1, 2, and 3 slits, respectively) were performed. The insertion mechanism may be the cranial micro-slit insertion method described herein. Insertion time following cranial and dural incisions was observed to be 20-40 s. For a single array of 529 microelectrodes, this corresponds to an average insertion rate of <40-80 ms per electrode over 0.48 cm² of cortical surface area.

The disclosed working example also illustrates the modularity and scalability of the disclosed neural interface devices. As discussed above, the arrays may be fabricated to facilitate alignment and modular assembly, so that 529-electrode modules can be joined to yield larger constructs covering larger portions of the cortical surface at the same density, without significantly increasing the complexity, risk, or time required for array insertion. In some cases, it is also possible to insert multiple arrays through a single slit. In vivo insertions of doubly connected modules (1,058 channels each over 0.96 cm² of cortical surface area), and in vitro insertions of quadruply connected modules (2,116 channels each covering 1.92 cm² of surface area) were performed. The use of a doubly connected module halves the effective average insertion rate, and in this manner have achieved effective speeds as fast as <20 ms per electrode in vivo. Similarly, use of larger arrays, such as 1,024-channel electrode array modules, increases electrode insertion rate and surface area coverage without substantially increasing surgical risk.

The disclosed working example also demonstrates the ability to interface with multiple anatomic and functional areas of the neocortex simultaneously, performing bilateral insertions over somatosensory cortex, as shown fluoroscopically and additional insertions over visual cortex.

Further, FIG. 16 shows a modular configuration of two 529-channel arrays (1,058 microelectrodes in total) situ on the cortical surface following a small frontal craniotomy in a Göttingen minipig (top left) and the same region of cortical surface is shown immediately following array removal (bottom left), demonstrating an intact pial layer and no damage to the brain. Further, safety was also assessed using standard, semi-quantitative histology techniques following 30-day chronic array implantation. Histologic analysis demonstrated microscopic preservation of the cortical surface architecture and no systematic differences between implanted and non-implanted control regions (top right and bottom right, respectively, hematoxylin and eosin).

In connection with the disclosed working example, the safety and reversibility of the disclosed neural interface device was also demonstrated. Four or more insertion sites were inspected immediately by craniectomies postmortem, following euthanasia, to assess the cortical surface for any gross or microscopic evidence of tissue damage immediately following array removal (“reversal” of implantation). The pia remained intact in all cases, and no evidence of tissue disruption at the cortical surface corresponding to array placement was found.

### Experimental Results—Neural Recording

Neural recording was performed in connection with the neural interfaces described herein.

In some embodiments, the disclosed systems were used for the free recording of spontaneous cortical activity. In particular, using the systems and methods described herein, spectrograms may be generated from data obtained at 20 kHz per channel, where spectral density is computed for a temporal resolution of 50 ms and frequency resolution of 17.5 Hz using a Hann window.

To demonstrate spatial correlation between pairs of electrodes, downsampled stimulus-free neural data may be separated into continuous 2 s segments. Within each segment, the Pearson correlation coefficient r may be computed for each pair of electrodes and associated to the corresponding physical electrode distance. The average r values across an array may be pooled across 25 segments and averaged for each electrode distance.

As illustrated in this working example, embodiments of the disclosed neural interfaces having microelectrode arrays may be configured to perform neural recording. Multichannel micro-electrocorticography may be performed by the neural interfaces described herein. The micro-electrocorticography may be used to record neural activity across the cortical surface, including to better characterize, understand and quantify the extent of correlation in neural activity across the cortical surface. The extent to which electrodes exhibit correlated activity may depend on the distance, the electrode size, and the frequency band interrogated. High-frequency activity approaching the kilohertz range may exhibit relatively uncorrelated activity even at short range, consistent with the coexistence of nearby but relatively uncoupled neurons. On the other hand, cortical activity at the lower frequencies analyzed in traditional scalp EEG reflects bulk field activity and tends to be correlated across larger areas of the cortical surface. The disclosed systems may be used to quantify these phenomena, using high-density arrays capable of spanning large regions of cortical surface. Thus, the disclosed systems may be designed with optimal electrode geometry and spacing for use in neural prostheses that depend on accurate decoding of neural activity.

### Experimental Results—Neural Stimulation and Neural Decoding

Neural stimulation was performed in connection with the neural interfaces described herein.

For example, somatosensory evoked potentials (SSEPs) are evoked by applying a periodic pressure stimulus at target locations in turn, including the rostrum, forelimbs, and hindlimbs. Neural response waveforms are temporally aligned to the stimulus onset. SSEPs are then computed as the averaged time-aligned signals over 50 stimuli.

To elicit visual evoked potentials (VEP), the eyelid corresponding to the stimulated retina is retracted temporarily while periodic 50 ms flashes are generated at 1 Hz from an array of white light-emitting diodes. Neural response waveforms are temporally aligned to the stimulus onset. VEPs are calculated as the time-aligned averaged signals over 100 trials.

Cortical stimulation in accordance with the disclosed embodiments may involve electrical stimulation at the cortical surface applied at one of the 200 μm electrodes, controlled by the Intan Technologies RHS controller and RHX software discussed above. Charge-balanced, biphasic, cathodic-first, 200 μs pulses of 100 μA peak current may be delivered at 0.25 Hz. The evoked potentials are recorded over a series of trials. During analysis, for each trial and electrode, 20 ms segments after variable delay (0, 2, 5, 10, 15, 20, 25, 30, 50 ms) post-stimulus are first Fourier transformed, then integrated for spectral power in the gamma band (30-100 Hz) and averaged over trials.

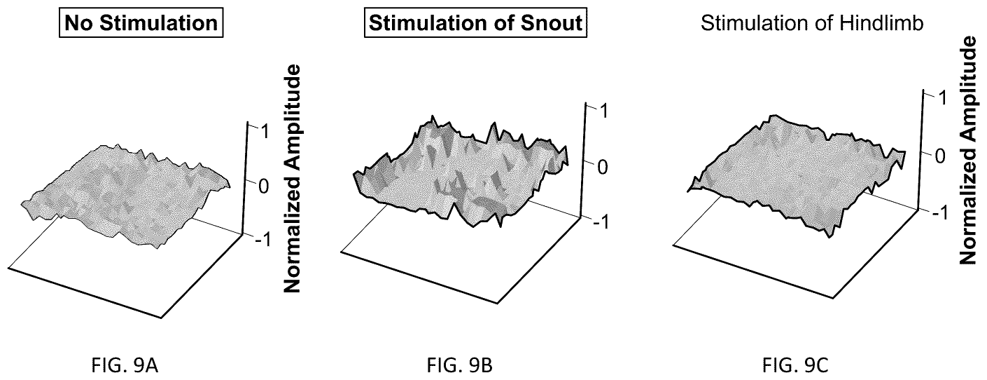

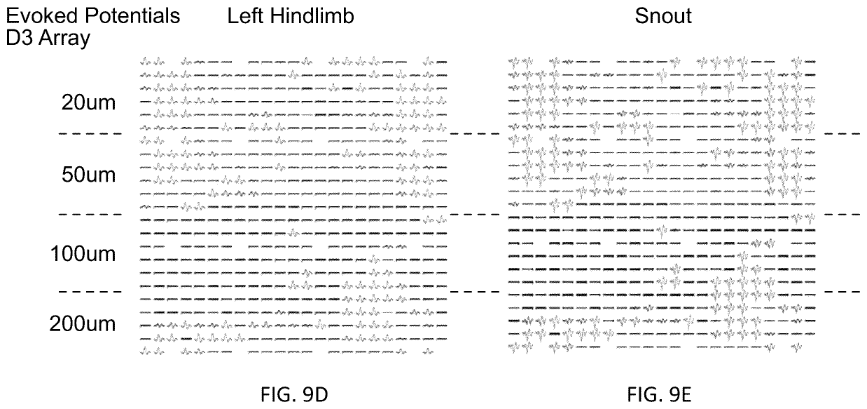

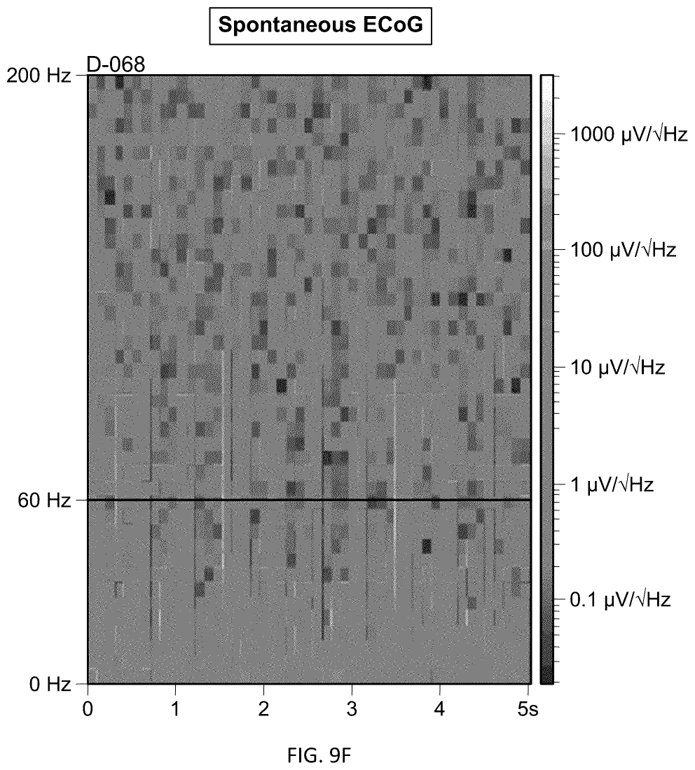

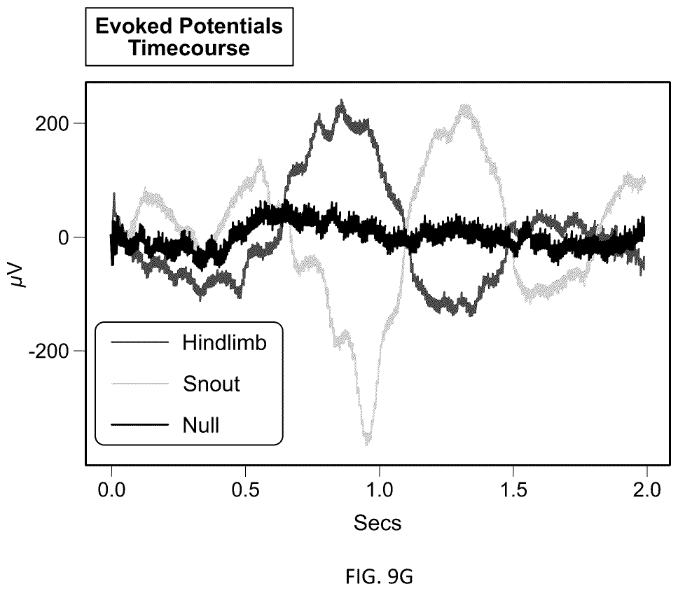

FIGS. 9A-9C illustrate brain responses in connection with no stimulation (FIG. 9A), stimulation of the snout (FIG. 9B), and stimulation of the hindlimb (FIG. 9C) by illustrating recordings across the electrode array in response to simulation.

FIG. 9D illustrates experimental data from stimulation, in accordance with embodiments of the present disclosure. For example, evoked potentials responsive to stimulation of the left hindlimb as observed across the plurality of electrodes on the microelectrode arrays is illustrated in FIG. 9D.

FIG. 9E illustrates experimental data from stimulation, in accordance with embodiments of the present disclosure. For example, evoked potentials responsive to stimulation of the snout as observed across the plurality of electrodes on the microelectrode arrays is illustrated in FIG. 9E.

Thus, this working example illustrates that embodiments of the disclosed neural interfaces having microelectrode arrays may be configured to perform neural stimulation. For example, the disclosed systems may provide targeted neurostimulation in connection with closed-loop brain-computer interfaces, as well as for neural prostheses for restoring functions such as vision. The disclosed systems may provide cortical stimulation from a thin-film cortical surface microelectrode that is configured to access the visual cortex in a minimally invasive fashion, electrophysiologically confirm array placement over functioning visual cortex, stimulate the cortical surface, and monitor stimulus-evoked cortical activity.

FIG. 9F illustrates experimental data from stimulation, in accordance with embodiments of the present disclosure. In particular, FIG. 9F illustrates recording of neural activity by the microelectrode array, and particularly the raw spectrum of responses to the evoked potentials.

FIG. 9G illustrates experimental data from stimulation, in accordance with embodiments of the present disclosure. In particular, FIG. 9G illustrates that the raw neural data can be decoded into distinct neural signatures corresponding to the different body regions (i.e., hindlimb, snout, no stimulus).

In some embodiments, neural decoding may be performed as follows. Single- and multi-channel, one-shot, binary classification decoding efficacy may be demonstrated with a template matching approach. Recording segments of 2 s duration may be classified as either an evoked potential or spontaneous activity. SSEPs from left hindlimb and right hindlimb may be taken as templates, and r is computed between the template and recording segment. SSEPs are computed over 30 trials as described previously, and template matching is tested over 20 SSEP trials and 20 segments of spontaneous activity. The receiver operating characteristic (ROC) curve and accuracy is then calculated by varying threshold r-value. Whole-array decoding operates similarly with the single-channel template replaced by an unweighted concatenation of all single-channel templates.

Thus, this working example illustrates that embodiments of the disclosed neural interfaces having microelectrode arrays may be configured to perform neural decoding. Signals across the brain, including those related to sensation, and vision, may be recorded and then decoded by the neural interfaces described herein. In some embodiments, the disclosed neural interfaces may be positioned such that the microelectrode arrays span across large surface areas. In some embodiments, the microelectrode arrays may be positioned in relation to the functional neuroanatomy of the brain. The disclosed neural interface may be configured such that the microelectrode arrays may be configured to access both hemispheres of the brain and/or multiple functional modalities (e.g., vision, somatic sensation). It is envisioned that neural decoding techniques may include approaches to parallel computation and machine learning techniques in optimizing the interpretation of array-based cortical signals, some techniques of which are described in further detail below.

### Experimental Results—Neural Recoding

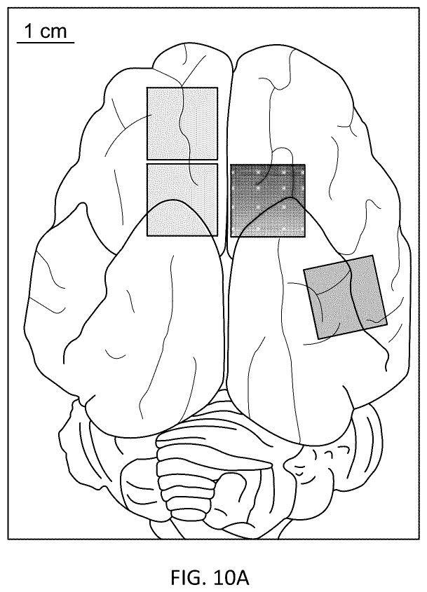

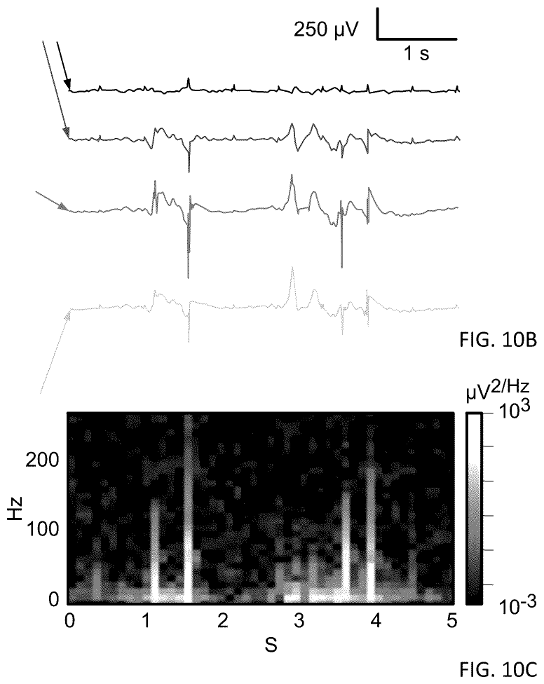

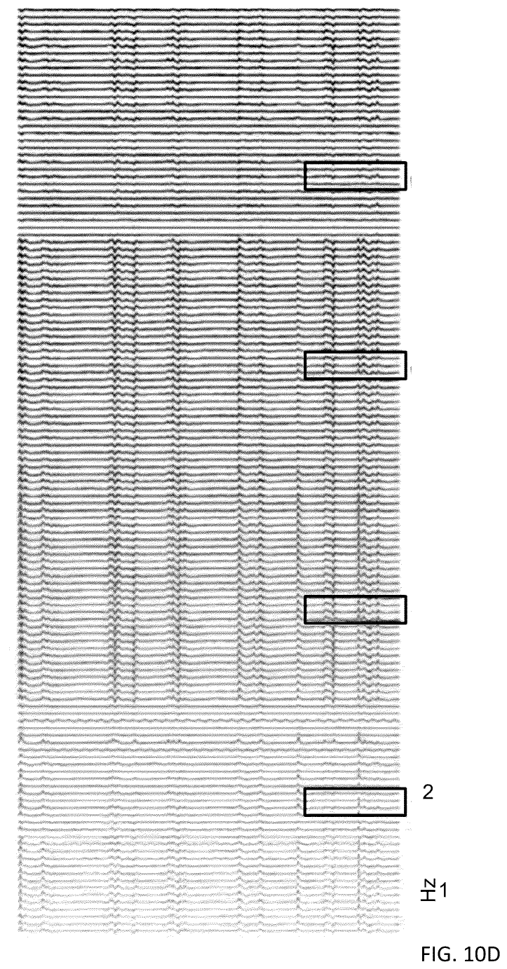

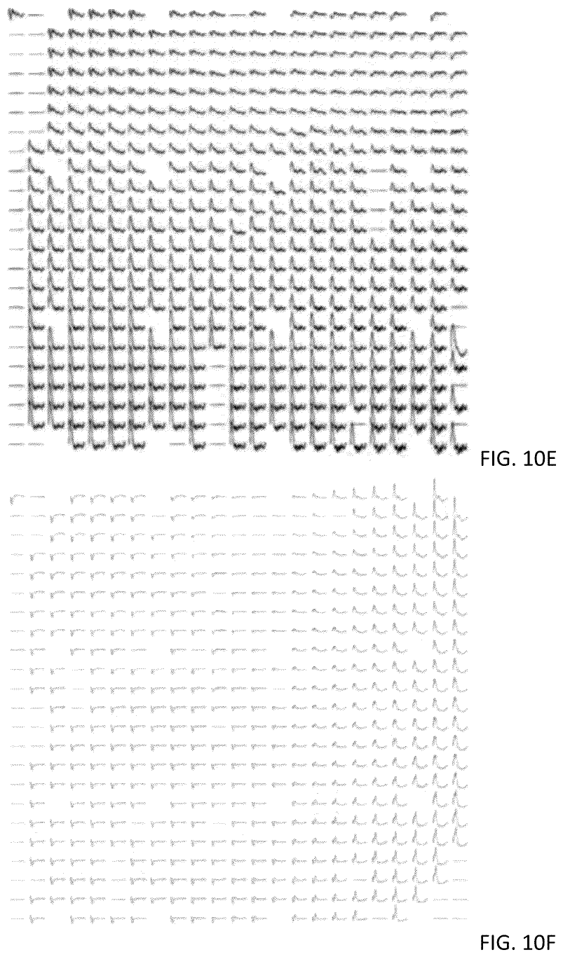

Recordings from the neural interface device in connection with spontaneous activity and evoked potentials were observed. For example, FIG. 10A provides an anatomic schematic of the Göttingen minipig brain showing areas indicating placement of subdural microelectrode array modules. FIG. 10B provides example recording traces and FIG. 10C provides an example spectrogram corresponding to the highlighted channels from an array over a somatosensory region of the right hemisphere as illustrated in FIG. 10A. FIG. 10D provides an illustration of recording of spontaneous neural activity from somatosensory cortex in a sample of channels from the highlighted 529-channel module. FIG. 10E provides an illustration of somatosensory evoked potentials corresponding to stimulation of the left hindlimb. FIG. 10F provides an illustration of visual evoked potentials corresponding to photostimulation of the left eye.

As illustrated in FIGS. 10A-10F, the neural devices described herein may be used for neural recording. For example, the illustrated implanted arrays may be used for multichannel neural recording using several paradigms, capturing both spontaneous cortical electrographic activity (FIG. 10D) as well as somatosensory and visual evoked potentials (FIGS. 10E and 10F). Similarly, visual evoked potentials may be recorded.

In the illustrated working example, during recording of spontaneous cortical activity, multiple 3-minute epochs were captured and analyzed. Electrocorticograms were obtained and reviewed in time- and frequency domains. Representative time traces and spectrograms are shown in FIGS. 10A-10F.

In the illustrated working example, evoked potentials were obtained across multiple arrays through time-locked stimulation paradigms. Robust array-based somatosensory evoked potentials were demonstrated in arrays positioned over somatosensory cortex, corresponding to stimulation of the hindlimbs, as well as the rostrum. Visual evoked potentials were also obtained in arrays positioned over visual cortex following time-synchronized photo-stimulation of the retina.

### Experimental Results—Neural Decoding

Neural decoding was performed in connection with the neural interfaces described herein. For example, data corresponding to decoding visual and somatosensory stimuli was obtained.

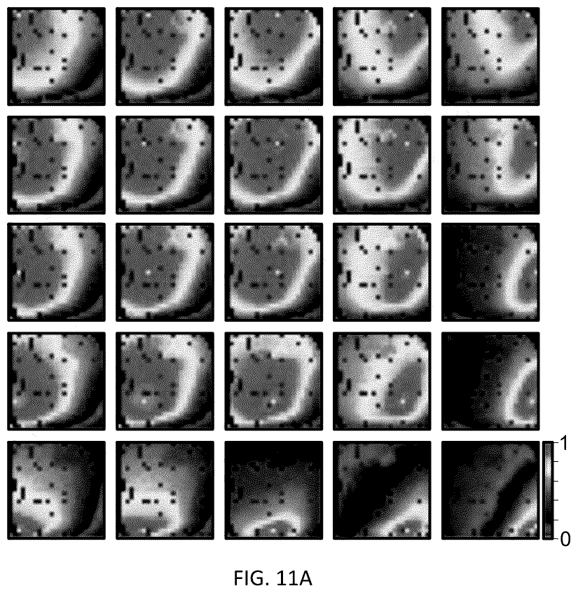

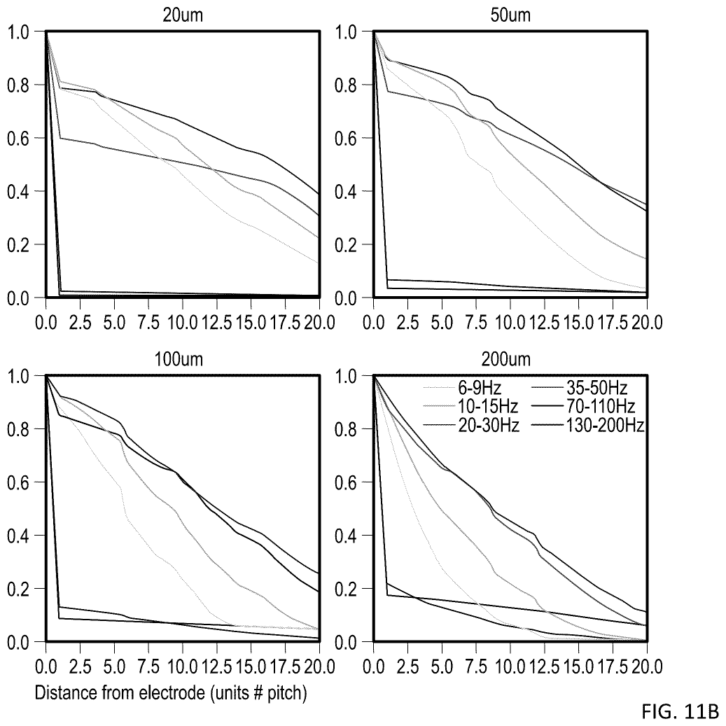

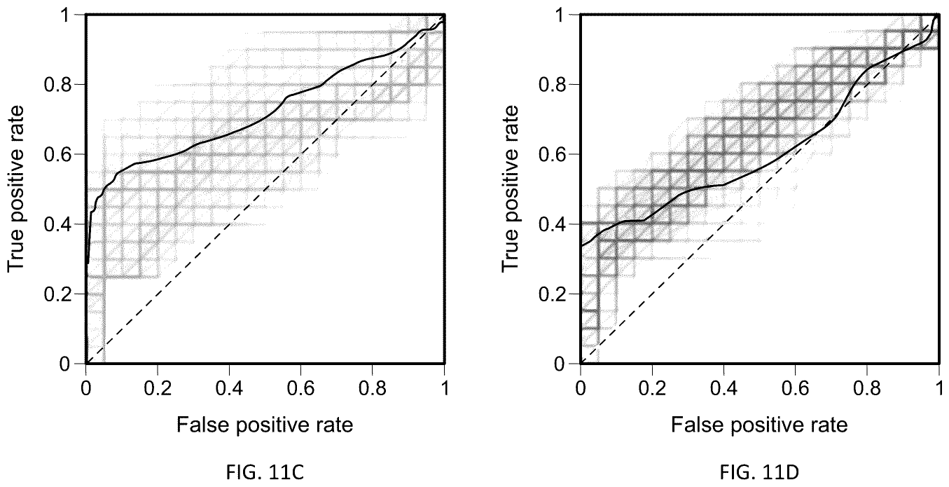

For example, FIGS. 11A-D illustrate experimental data including recordings from closely spaced microelectrodes across a single channel array. FIGS. 11A-D illustrate the level of correlated or uncorrelated information from the recorded neural areas.

FIG. 11A provides a correlation map of the Pearson correlation coefficient r² computed for signals recorded during spontaneous cortical activity, with correlations across the entire array in each sub-plot referenced to one of 25 electrodes (grey dots) distributed evenly or uniformly over the array, as represented by the array of sub-plots. In some embodiments, the intra-electrode spacing can be less 400 microns. In one illustrative embodiment, the intra-electrode spacing can be approximately 330 microns. FIG. 11B illustrates the dependence of correlation on electrode separation as a function of electrode size and spectral band. FIGS. 11C and 11D illustrate characteristics of the accuracy and robustness of neural decoding from right visual cortex. Shown are the receiver operating characteristic (ROC) curves for decoding visual stimulation of the left (FIG. 11C) or right (FIG. 11D) eye. Colored traces represent the ROC curves from template matching individual channels, while the black trace represents decoding performance obtained by integrating information from all channels into a collective template (averaged in Monte Carlo fashion over 20 shuffles of the training and testing datasets).

As illustrated in FIGS. 11A-11C the working example provides insights into the utility of this system for neural decoding by both spontaneous activity and evoked potentials. FIGS. 11A-11C illustrate the degree to which spontaneous neural activity, as recorded from reference sites sampled from across a given array, is correlated with activity simultaneously recorded from other sites. The degree of correlation decreases with distance as illustrated in FIG. 11A and with increasing frequency as illustrated in FIG. 11B.

Importantly, even closely spaced electrodes exhibit incompletely correlated activity, particularly at higher frequencies. For example, broadband r-squared is in the range of 0.8 at 300 μm spacing for 20 μm electrodes.

These properties of the sampled electrocorticographic activity, together with the ability to record robust evoked potential responses across hundreds of channels, may facilitate straightforward and highly accurate neural decoding of somatosensory stimuli, as summarized in FIGS. 11C and 11D.

The working example used a straightforward, unweighted template-matching algorithm as the basis for distinguishing stimulation of the left or right hindlimb from absence of stimulation (free recording). Multichannel decoding using the large number of electrodes available from the entire array was both more robust and more accurate that decoding using any single electrode, with accuracies reaching 73% on the left and 67% on the right in off-line decoding.

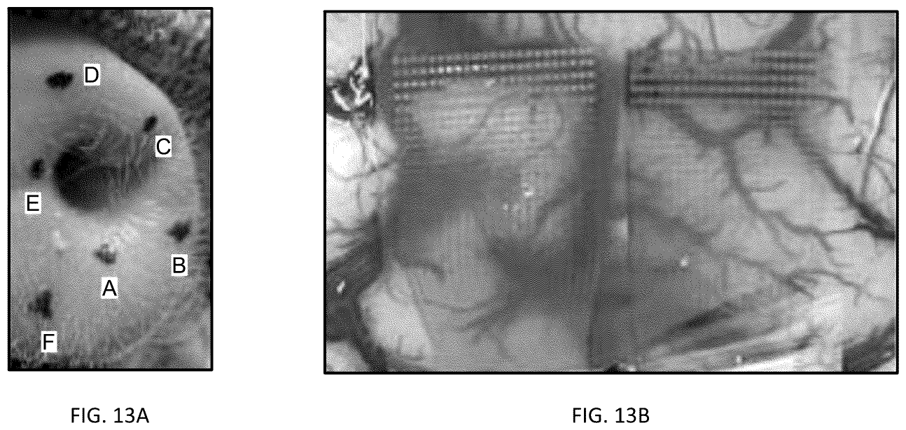

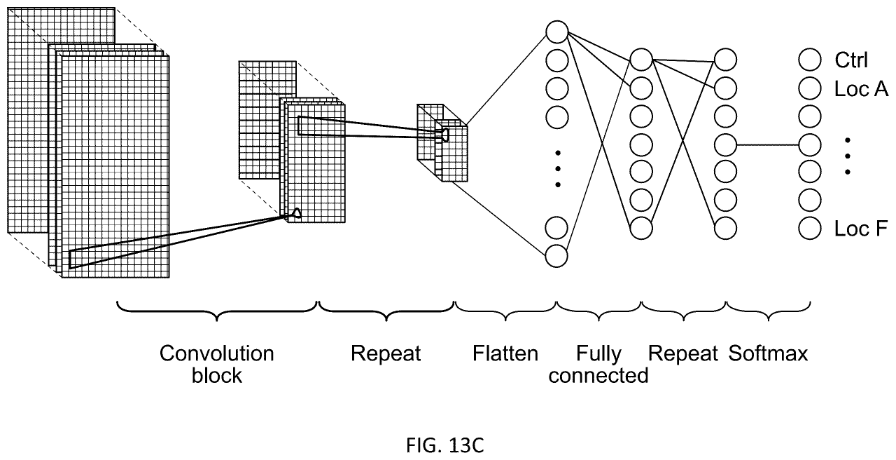

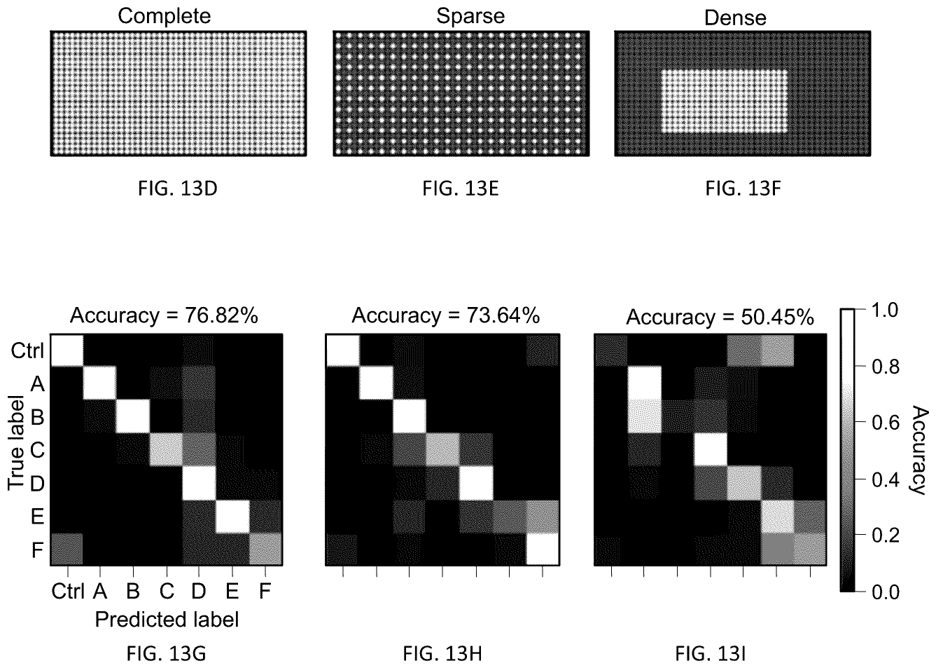

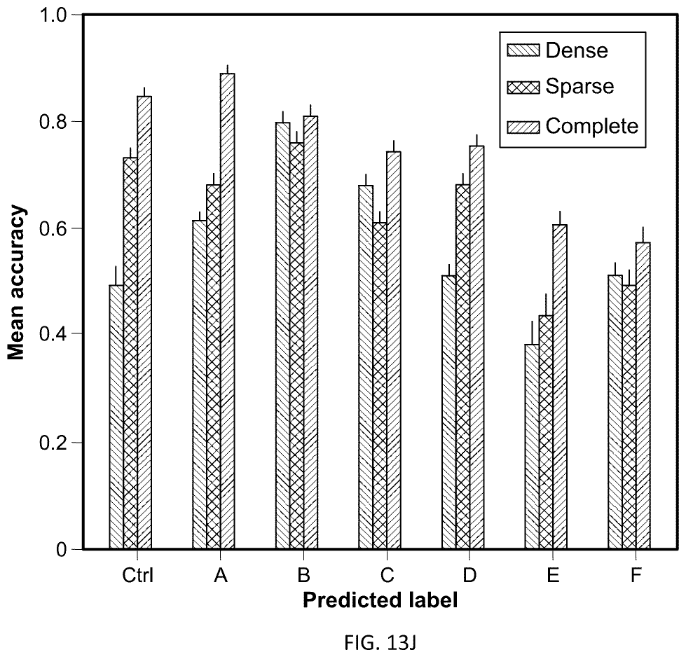

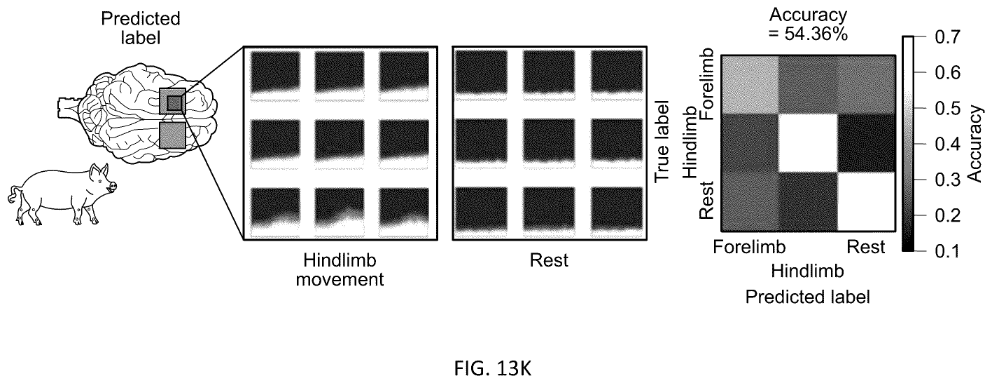

As another example, FIGS. 13A-G illustrate additional experimental data. In particular, FIG. 13A shows tactile stimulation locations on the rostrum of the Göttingen minipig used for sensory discrimination and neural decoding and FIG. 13B shows the placement of two electrode arrays (1058 microelectrodes total) on the cortical surface overlying the rostrum somatosensory cortex.

Machine learning and neural network techniques were used for decoding and analyzing the data captured during these experiments. In particular, FIG. 13C shows the baseline architecture of the convolutional neural network used for decoding stimulated location in these experiments. A convolution block consists of the following steps in sequence: 3×3 convolutions, leaky ReLU, dropout during training, “max pooling” with stride 2, and batch normalization. Each fully connected layer is followed by leaky ReLU.

FIGS. 13D-I show different electrode arrangements used in these experiments and the corresponding confusion matrices for those electrode arrangements. In particular, FIG. 13D shows a configuration where all electrodes are recruited in decoding (i.e., a “complete grid”) and FIG. 13G shows the corresponding confusion matrix for decoding with the complete grid. FIG. 13E shows a configuration where every other electrode in both spatial dimensions is recruited for decoding (i.e., “sparse grid”), totaling 23×12 electrodes and FIG. 13H shows the corresponding confusion matrix for decoding with the sparse grid. FIG. 13F shows a confusion where a dense subset of a 23×12 grid of adjacent electrodes (i.e., “dense grid”) is recruited for decoding, resulting in a data rate equivalent to using a sparse grid, and FIG. 13I shows the corresponding confusion matrix for decoding with the dense grid. FIG. 13J shows the decoding accuracies for each tactile stimulation location and control, using convolution kernels emphasizing different options provided by the high-density cortical surface arrays (complete coverage at full density, sparse density, and subsampled partial coverage). Error bars represent the standard error of the mean for 25 models trained per grid configuration.

FIG. 13K illustrates a schematic showing bilateral placement of 1024-channel electrode arrays (2048 microelectrodes total) for motor decoding during volitional walking (inset schematic). Further, illustrative subsamples of spectrograms drawn from the same group of electrodes across the array (the highlighted region in the schematic) are shown, which correspond to each of two limb movement states, i.e., moving and stationary. The spectrograms demonstrate distinct, state-dependent patterns of cortical activity. Further, a confusion matrix for decoding limb movement and rest state using pre-movement neural activity with a convolutional neural network (CNN) (10-fold cross-validation) is also shown.

### Experimental Results—Neural Stimulation

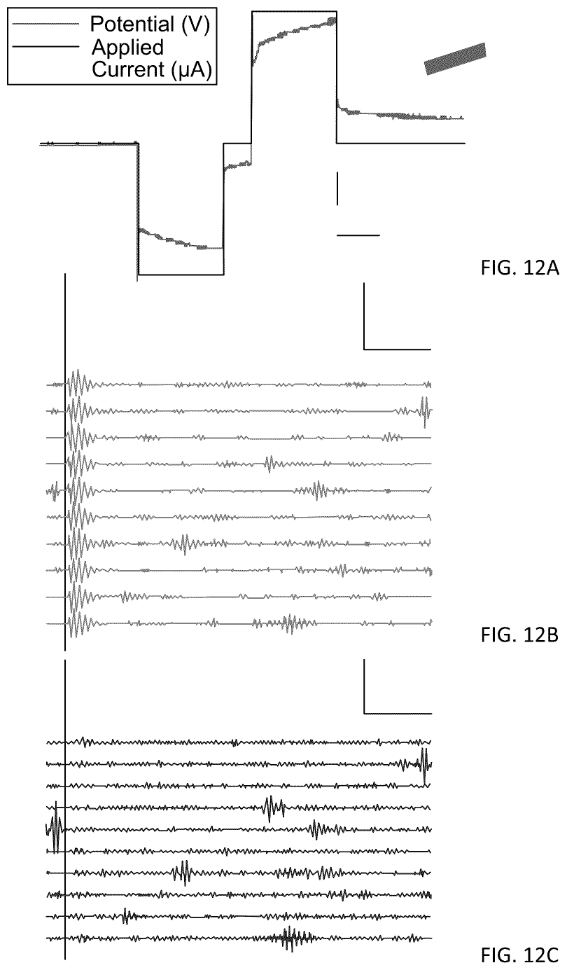

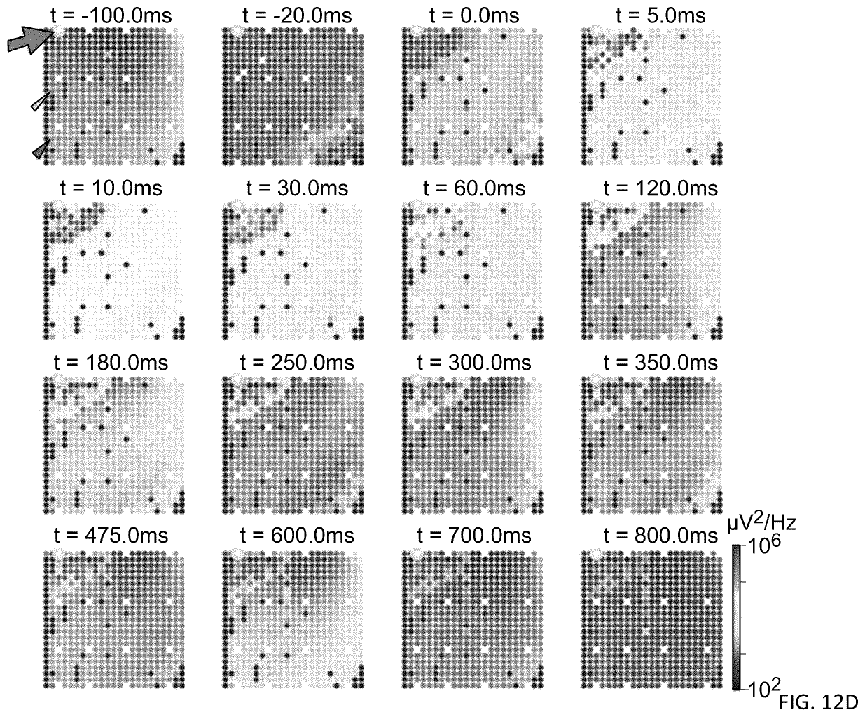

Neural stimulation experiments were performed in connection with the neural interfaces described herein. Electrode arrays built in accordance with the systems described herein are capable of bidirectional function, with every electrode able to perform either recording or stimulation. In one working example, for the described set of in vivo experiments, 16 electrodes per array were designated for use in cortical stimulation. As demonstrated in FIGS. 12A-12D, safe stimulation thresholds were determined in vitro for each electrode type. Briefly, the water window was determined by CV measurement to be approximately 1.85 V, and biphasic pulses were applied at 50 Hz to a test electrode within the range of anticipated stimulation parameters to confirm safe stimulation. A total of 50,000 pulses of 10 nC per phase and 100,000 pulses of 20 nC per phase (50 and 100 μA respectively with a 200 μs pulse width) were found to cause no change in impedance on the test electrode, so stimulation currents in this range were determined to be acceptable for the 30 pulses per electrode used in vivo. Cortical stimulation was performed in vivo, with a single 200 μm electrode used for stimulation in each trial, and the remaining sites on the same array, as well as all sites on adjacent arrays, were used for recording.

After characterizing a safe operating regime for cortical stimulation, focal stimulation of the visual cortex using the paradigm described was performed, while monitoring stimulus-evoked cortical activity. Cortical stimulation evoked an increase in high-gamma-band power across the surrounding region monitored by the array, lasting approximately 1 s. The most highly activated region formed an annulus of approximately 1 mm around, but not immediately surrounding the stimulating electrode. Regions farther from the annulus exhibited a transient reduction in cortical activation during this period. No induced saccades were observed during stimulation of the visual cortex. An example recording of the cortical electrophysiologic response to cortical stimulation is shown in FIGS. 12A-12D.

In particular an example trace of the stimulation waveform is shown in FIG. 12A as generated by a stimulating electrode.

FIG. 12B illustrates example traces for an electrode near the stimulation electrode (red circle) band-pass filtered for 70-150 Hz (gamma band) from 10 stimulation trials recording from one microelectrode near the stimulation site (orange ring). These trials reveal increased cortical local field activity immediately following cortical stimulation for regions near the stimulating electrode. Dotted vertical line indicates the stimulus. FIG. 12C illustrates corresponding traces for an electrode farther from the stimulus (blue ring). While still being responsive to stimulation, more distant regions exhibit less evoked activity. FIG. 12D illustrates the time progression of high-gamma-band power across the array of microelectrodes. Stimulation starts at t=0 μs and ends at t=400 μs.

Referring now to FIGS. 14A-J, there are shown additional data from neural stimulation experiments were performed in connection with the neural interfaces described herein. These experiments were performed to demonstrate that cortical microstimulation can modulate cortical activity in ways that can be characterized in high spatial and temporal detail. In particular, FIG. 14A shows a stimulation waveform used for in vitro confirmation of safe polarization potential, with 100 μA overlaid on the waveform for reference. In vivo applied current waveforms used the same applied current but without the interphase delay used for identifying polarization potential. FIG. 14B shows example traces for an electrode (indicated with an arrow in FIG. 14G) near the stimulation electrode (indicated with a ring in FIG. 14G) for 8 stimulation trial recordings. The “activity” of each trial is computed as the variance of the signal from 200 ms to 2000 ms post-stimulation (green box), and the average activity is taken over 40 trials. FIG. 14C shows corresponding traces for the animal under heavier anesthesia and FIG. 14D shows corresponding traces for the electrode without stimulation under light anesthesia.

FIGS. 14E and 14F illustrate stimulated activity plotted against control activity. Each point represents an individual microelectrode and the highlighted points are located within 5 electrode spacings (radial) from the stimulating electrode. The histograms show the distributions of activity with (side panel) and without (top panel) stimulation. Arrow shows the same electrode as indicated in panel in FIG. 14G. Further, FIG. 14F shows stimulated activity under light versus heavy anesthesia, plotted using a scheme analogous to that used for FIG. 14E. The histograms show the distributions of stimulation-induced activity under different levels of anesthesia.

FIG. 14G shows activity across the two adjacently placed arrays with stimulation applied at the highlighted electrode. FIG. 14H shows activity across the array without stimulation, using the same color scale as in FIG. 14G. FIG. 14I shows differential activity across the arrays, calculated as the difference between FIGS. 14G and H, revealing a region of suppressed activity surrounding the stimulating electrode and extending across two adjacent arrays. FIG. 14J shows a map of differential stimulated activity between light and heavy anesthesia.

Although the working examples and experimental results described herein are related to an animal model, Gottingen mini-pigs, it is envisioned that the techniques, methods, systems and apparatus discussed herein may be adapted for use in other animals, including humans.

1. A neural interface for intradural implantation, the neural interface comprising:
a flexible substrate, wherein the flexible substrate comprises a first bioinert material;
a pocket disposed on a first side of the flexible substrate; and
a plurality of modules disposed on a second side of the flexible substrate, wherein the second side opposes the first side, wherein each of the modules comprises a plurality of microelectrodes, wherein the modules are removably connected together, and wherein the microelectrodes do not penetrate a surface of a brain against which the microelectrodes are positioned and comprise a second bioinert material.

2. The neural interface of claim 1, wherein:
each of the modules comprises proximal holes and distal holes; and
the modules are removably coupled together by aligning the proximal holes of a preceding module with the distal holes of a succeeding module of the plurality of modules.

3. The neural interface of claim 2, wherein an adhesive is applied to an overlapping region of the proximal holes of the preceding module and the distal holes of the succeeding module of the plurality of modules.

4. The neural interface of claim 3, wherein the adhesive comprises cyanoacrylate.

5. The neural interface of claim 1, wherein the modules are removably couplable together by aligning a first alignment guide in a preceding module with a second alignment guide in a succeeding module of the plurality of modules.

6. The neural interface of claim 1, wherein the electrodes that are distributed evenly in the microelectrode arrays.

7. The neural interface of claim 1, wherein the electrodes have a diameter of 20-200 μm.

8. The neural interface of claim 1, wherein the electrodes are configured for at least one of recording or stimulation.

9. The neural interface of claim 1, wherein each of the plurality of microelectrode arrays comprises 1,024 channel arrays.

10. The neural interface of claim 1, wherein the first bioinert material comprises a polyimide and the second bioinert material comprises at least one of titanium or platinum.

11. A surgical system comprising:
an insertion paddle; and
a neural interface for intradural implantation, the neural interface comprising:
a flexible substrate, wherein the flexible substrate comprises a first bioinert material;
a pocket for receiving the insertion paddle, the pocket disposed on a first side of the flexible substrate; and
a plurality of modules disposed on a second side of the flexible substrate, wherein the second side opposes the first side, wherein each of the modules comprises a plurality of microelectrodes, wherein the modules are removably connected together, and wherein the microelectrodes do not penetrate a surface of a brain against which the microelectrodes are positioned and comprises a second bioinert material.

12. The surgical system of claim 11, wherein:
each of the modules comprises proximal holes and distal holes; and
the modules are removably coupled together by aligning the proximal holes of a preceding module with the distal holes of a succeeding module of the plurality of modules.

13. The surgical system of claim 12, wherein an adhesive is applied to an overlapping region of the proximal holes of the preceding module and the distal holes of the succeeding module of the plurality of modules.

14. The surgical system of claim 13, wherein the adhesive comprises cyanoacrylate.

15. The surgical system of claim 11, wherein the modules are removably coupled together by aligning a first alignment guide in a preceding module with a second alignment guide in a succeeding module of the plurality of modules.

16. The surgical system of claim 11, wherein the electrodes that are distributed evenly in the microelectrode arrays.

17. The surgical system of claim 11, wherein the electrodes have a diameter of 20-200 μm.

18. The surgical system of claim 11, wherein the electrodes are configured for at least one of recording or stimulation.

19. The surgical system of claim 11, wherein each of the plurality of microelectrode arrays comprises 1,024 channel arrays.

20. The surgical system of claim 11, wherein the insertion paddle comprises a semi-rigid material.

21. The surgical system of claim 11, wherein the first bioinert material comprises a polyimide and the second bioinert material comprises at least one of titanium or platinum.
